(19) United States
(12) Patent Application Publication
(10) Pub. No.: US 2023/0278957 A1
(43) Pub. Date: Sep. 7, 2023

(54) TREX1 INHIBITORS AND USES THEREOF

(71) Applicant: WAKE FOREST UNIVERSITY HEALTH SCIENCES, Winston-Salem, NC (US)

(72) Inventors: Fred W. PERRINO, Winston-Salem, NC (US); Thomas HOLLIS, Winston-Salem, NC (US); Scott E. HARVEY, Winston-Salem, NC (US); Wayne O. HEMPHILL, Winston-Salem, NC (US)

(21) Appl. No.: 18/040,192

(22) PCT Filed: Aug. 3, 2021
(86) PCT No.: PCT/US2021/044253

§ 371 Date: Feb. 1, 2023

Related U.S. Application Data
(60) Provisional application No. 62/706,167, filed on Aug. 4, 2020.

(51) Int. Cl. C07D 209/48; C07C 309/44; C07D 417/12; C07D 405/14; C07D 413/14; C07D 405/12; C07D 513/14; C07D 471/14

(52) U.S. Cl. CPC C07D 209/48; C07C 309/44; C07D 405/12; C07D 405/14; C07D 413/14; C07D 417/12; C07D 471/14; C07D 513/14

(57) ABSTRACT

Active compounds of Formulas I - IX are provided herein that are useful as TREX1 inhibitors, which in turn is useful for stimulating the cGAS-STING pathway. As such, these compounds may be used in a method of treating disease or disorder in which modulation of TREX1 would be of benefit, such as a cancer, viral infection, or autoimmune disease.

### CROSS-REFERENCE TO RELATED APPLICATIONS

This application claims the benefit of U.S. Provisional Pat. Application No. 62/706,167, filed Aug. 4, 2020, the disclosure of which is incorporate by reference herein in its entirety.

### BACKGROUND

As part of its role in the defense against pathogens, the innate immune system utilizes sensors called pattern recognition receptors (PRRs). When a pathogen is encountered, pathogen-associated molecular patterns (PAMPs) will activate these PRRs, which initiate an immune response. PRRs can also be activated in the absence of pathogens by damage/danger-associated molecular patterns (DAMPs). Interestingly, PRRs will sometimes respond to DNA or RNA as a DAMP/PAMP during a process known as nucleic acid sensing¹. In the DNA-sensing (DS) pathway cGAS-STING, binding of dsDNA to the receptor cGAS causes synthesis of a 2′-3′-cyclic GMP-AMP (cGAMP)², which in turn activates the endoplasmic reticulum-associated protein STING (STimulator of INterferon Genes)^{3,4}. Upon activation, STING traffics to the Golgi apparatus where it recruits Tank Binding Kinase 1 (TBK1) to phosphorylate it⁵. Phosphorylated STING recruits interferon regulatory factor 3 (IRF3) for phosphorylation by TBK1, and activated IRF3 then dimerizes and translocates to the nucleus to promote expression of type-I interferons (IFN)⁶. After its expression, binding of IFN to its receptor (IFNAR) induces immune activation by promoting the proliferation and maintenance of natural killer (NK) and memory CD8⁺ T cells, stimulating dendritic cells (DC), and more broadly by increasing the expression of interferon-stimulated genes (ISGs)⁷. The cGAS-STING pathway has been proposed to act as a broad NAS pathway for many sources of DNA⁶, though a complete list of insulting nucleic acids has not been definitively established. Perhaps unsurprisingly, the cGAS-STING pathway has also been proposed to play a role in the tumor microenvironment, where it senses tumor-derived DNA from irradiated cancer cells and promotes IFN-dependent antitumor immunity^{8,9}.

The exonuclease TREX1 (Three-prime Repair EXonuclease) is now recognized as a gatekeeper to the cGAS-STING pathway. TREX1 is a homodimeric 3′ -> 5′ exonuclease that exhibits activity on dsDNA and ssDNA substrates in vitro, and displays perinuclear localization in cells¹⁰⁻¹². Each protomer of the TREX1 homodimer is composed of an N-terminal (1-242) catalytic domain responsible for exonuclease activity and inter-protomer contacts, plus a C-terminal (243-314) tail region responsible for the enzyme’s localization and known protein-protein interactions¹⁰⁻¹⁴. The TREX1 protomers do not dissociate at measurable rates, and this obligate dimeric structure is unique among exonucleases¹⁵⁻²². This highly stable dimeric structure is mediated by an extensive inter-protomer hydrogen-bonding network including backbone contacts between β-strands, side-chain pairings, and hydrophobic packing of antiparallel helices¹⁵⁻²². Additionally, complex interactions across TREX1′s dimer interface mediate contribution by one protomer to catalysis in the opposing protomer¹⁸, and raise the possibility of correlated activity between the two active sites. TREX1′s exonuclease activity is dependent on the presence of a 3′-hydroxyl on the deoxyribonucleic acid substrate, and thus DNA with 3′-modifications are not viable TREX1 substrates^{10,23-26}. TREX1 also requires two magnesium ions that help coordinate substrate binding in the active site of each protomer¹⁰.

In humans, mutations in TREX1 have been causatively linked to a spectrum of autoimmune conditions like Aicardi-Goutières syndrome (AGS), familial chilblain lupus (FCL), and retinal vasculopathy with cerebral leukodystrophy (RVCL), and associated with systemic lupus erythematosus (SLE)²⁷. Multiple TREX1 mutant mouse models have been generated, which also develop similar autoimmune phenotypes to those seen in humans²⁸⁻³¹. In humans and mice, TREX1 inactivity stimulates the cGAS-STING pathway to promote an IFN-response, which in turn drives associated pathology³²⁻³⁴. While TREX1′s biological substrate(s) are still being investigated^{20,21,32}, the specificity of cGAS suggests that the TREX1 substrate relevant to chronic immune activation is dsDNA³⁵. Thus, TREX1 functions in vivo to degrade dsDNA and prevent aberrant DNA-sensing through the cGAS-STING pathway.

TREX1, like cGAS-STING, has been proposed to function in the tumor microenvironment by degrading tumor-derived immunogenic DNA from drug or radiation-treated cancer cells, consequently preventing activation of cGAS-STING³⁶⁻³⁹. This model of TREX1, cGAS-STING, and the tumor microenvironment suggests that activation of the cGAS-STING pathway, either directly or through inhibition of TREX1 exonuclease activity, would confer antitumor immunity. As a result, activation of the cGAS-STING pathway is a developing area of immunotherapy, and several candidate STING agonists have already reached clinical trials after showing promise in human cells and animal models⁴⁰⁻⁴⁴. Collectively, studies to date identify TREX1 inhibition as a novel approach to activate the cGAS-STING pathway and promote antitumor immunity.

The only report of TREX1 inhibitors to date, however - Qian, J. Discovery of small-molecule compounds targeting TREX1 : University of Southern California Dissertations and Theses (2012) - identified six compounds of IC₅₀ ~ 18-36 µM, and little information beyond initial potency was provided. New TREX1 inhibitors with accompanying characterization are needed.

### SUMMARY

Active compounds are provided herein that are useful as TREX1 inhibitors, which in turn is useful for stimulating the cGAS-STING pathway. As such, these compounds may be used in a method of treating disease or disorder in which modulation of TREX1 would be of benefit, such as a cancer, viral infection, or autoimmune disease.

In some embodiments, the method includes administering to a subject in need thereof a therapeutically effective amount of a compound of Formula I, a compound of Formula II, a compound of Formula III, a compound of Formula IV, a compound of Formula V, or a pharmaceutically acceptable salt or prodrug thereof, as defined herein.

In some embodiments, the method includes administering to a subject in need thereof a therapeutically effective amount of a compound of Formula VI, a compound of Formula VII, a compound of Formula VIII, or a compound of Formula IX, or a pharmaceutically acceptable salt or prodrug thereof, as defined herein.

In some embodiments, the administration may include, but is not limited to, administering a pharmaceutical composition comprising said compound, pharmaceutically acceptable salt or prodrug thereof.

Also provided herein according to some embodiments is a compound of Formula I, a compound of Formula II, a compound of Formula III, a compound of Formula IV, a compound of Formula V, a compound of Formula VI, a compound of Formula VII, a compound of Formula VIII, or a compound of Formula IX, as defined herein, or a pharmaceutically acceptable salt or prodrug thereof.

Further provided is a pharmaceutical composition comprising a compound of Formula I, a compound of Formula II, a compound of Formula III, a compound of Formula IV, a compound of Formula V, a compound of Formula VI, a compound of Formula VII, a compound of Formula VIII, or a compound of Formula IX, as defined herein, or a pharmaceutically acceptable salt or prodrug thereof. In some embodiments, the pharmaceutical composition is formulated for oral administration. In some embodiments, the pharmaceutical composition may include, but is not limited to, a capsule, cachet, lozenge, or tablet.

In some embodiments, the pharmaceutical composition is formulated in unit dosage form from 1 mg to 10 g of a compound of Formula I, a compound of Formula II, a compound of Formula III, a compound of Formula IV, a compound of Formula V, a compound of Formula VI, a compound of Formula VII, a compound of Formula VIII, or a compound of Formula IX, as defined herein, or a pharmaceutically acceptable salt or prodrug thereof.

Also provided herein is the use of a compound of Formula I, a compound of Formula II, a compound of Formula III, a compound of Formula IV, a compound of Formula V, a compound of Formula VI, a compound of Formula VII, a compound of Formula VIII, or a compound of Formula IX, as defined herein, or a pharmaceutically acceptable salt or prodrug thereof, for controlling or inhibiting TREX1, in a human or non-human animal subject in need thereof, or for the preparation of a medicament for controlling or inhibiting TREX1, in a human or non-human animal subject in need thereof.

Also provided herein is the use of a compound of Formula I, a compound of Formula II, a compound of Formula III, a compound of Formula IV, a compound of Formula V, a compound of Formula VI, a compound of Formula VII, a compound of Formula VIII, or a compound of Formula IX, as defined herein, or a pharmaceutically acceptable salt or prodrug thereof, for stimulating the cGAS-STING pathway, or in the preparation of a medicament for stimulating the cGAS-STING pathway, in a human or non-human animal subject in need thereof.

Also provided herein is the use of a compound of Formula I, a compound of Formula II, a compound of Formula III, a compound of Formula IV, a compound of Formula V, a compound of Formula VI, a compound of Formula VII, a compound of Formula VIII, or a compound of Formula IX, as defined herein, or a pharmaceutically acceptable salt or prodrug thereof, for treating a cancer, or in the preparation of a medicament for treating a cancer, in a human or non-human animal subject in need thereof.

Also provided herein is the use of a compound of Formula I, a compound of Formula II, a compound of Formula III, a compound of Formula IV, a compound of Formula V, a compound of Formula VI, a compound of Formula VII, a compound of Formula VIII, or a compound of Formula IX, as defined herein, or a pharmaceutically acceptable salt or prodrug thereof, for treating a viral infection, or in the preparation of a medicament for treating a viral infection, in a human or non-human animal subject in need thereof.

Also provided herein is the use of a compound of Formula I, a compound of Formula II, a compound of Formula III, a compound of Formula IV, a compound of Formula V, a compound of Formula VI, a compound of Formula VII, a compound of Formula VIII, or a compound of Formula IX, as defined herein, or a pharmaceutically acceptable salt or prodrug thereof, for treating an autoimmune disease, or in the preparation of a medicament for treating an autoimmune disease, in a human or non-human animal subject in need thereof.

### BRIEF DESCRIPTION OF THE DRAWINGS

FIG. 1: Determining IC₅₀ of Candidate Compounds. Panels A-D: Example IC₅₀ Data of the Two Most Potent Hit Compounds. Standard time-course reactions were prepared in 150 µL volumes containing vehicle or indicated concentrations of inhibitor (Panel A= 0.1-10 µM; Panel C= 1-50 µM), incubated 1 hr at room temperature, and 20 µL samples of each reaction taken at time-points of 0, 5, 10, 20, 30, 45, & 60 minutes and quenched in 20 µL of 15X SYBR Green. Fluorescence was measured, and fluorescence versus time plots were normalized to maximal initial fluorescence and background fluorescence. Plots were fitted with nonlinear regression, then initial velocities of each reaction interpolated and normalized to vehicle-only control reactions to give percent activity. Plots of inhibitor concentration versus percent activity were fit with nonlinear regression to interpolate compound IC₅₀. Plots were generated in Prism (GraphPad) and combined in PowerPoint. Active compounds had IC₅₀ determined in at least three separate experiments. Panels E-F: Gel Assay Validation of Hit Compound IC₅₀. Several hit compounds, including those shown, had their IC₅₀ confirmed via gel assay to validate efficacy of the fluorescence-based biochemical assay. Results of the gel and fluorescence assays were in agreement for all compounds tested. Standard time-course reactions containing nicked plasmid were prepared in 150 µL volumes with the indicated concentrations of inhibitor, and incubated at room temperature. Reaction samples were taken at indicated times, quenched, dried in vacuo, resuspended, then visualized by agarose gel electrophoresis. Control lanes containing supercoiled dsDNA plasmid (‘Plasmid’) and undegraded nicked dsDNA plasmid (‘Nicked’) are included. Low-MW residual bands are ssDNA plasmid leftover from TREX1 exonuclease activity.

FIG. 2: Counter-Screening Hit Compounds Against EhTRXR^{WT}. Time-course reactions were prepared as described with the indicated concentrations of inhibitor (1-50 µM) or vehicle. Product formation was monitored via A412 readout in 35 s intervals over ~15 min. Plots of A412 versus time were fit with one-phase association nonlinear regression. Plots were generated in Prism (GraphPad), and combined in PowerPoint. Panel A: Positive control reactions with the known EhTRXR inhibitor Auranofin. Panels B-F: Counter-screens of several hit compounds; all hit compounds shown were inactive against EhTRXR.

FIG. 3: Counter-Screening Hit Compounds Against mTREX1 and hTREX2 Enzymes. Panel A: Summary chart of lead compounds’ IC₅₀ against hTREX1 vs mTREX1 versus hTREX2. Panels B-F: Overlay of lead compounds’ dose-response curves for hTREX1, mTREX1, and hTREX2. Dose-response curves were generated as described earlier. Each dose-response curve is representative of three IC₅₀ experiments. Graphs were prepared in Prism (GraphPad), and figure was assembled in PowerPoint.

FIG. 4: Reversibility of Inhibition for Lead Compounds. Panel A: Summary chart of the results for each lead compound. IC₅₀ ratios were calculated by dividing the ‘enzyme pre-incubation’ IC₅₀ by the ‘reaction pre-incubation’ IC₅₀. An IC₅₀ ratio of 10 is expected for irreversible inhibitors, and the results were classified as follows: ≥ 5.0 → Irreversible, 2.0-5.0 → Ambiguous, 0.5-2.0 → Reversible. Panels B-G: Standard IC₅₀ experiments were conducted with various concentrations of each lead compound. However, before enzyme addition to the reaction components the inhibitor or vehicle was pre-incubated with either the reaction components at ~1X the final concentration (‘Reaction Pre-Incubation’) or the enzyme at ~10X the final concentration (‘Enzyme Pre-Incubation’). The enzyme and reaction components were then combined to initiate reactions, and IC₅₀ calculated as discussed earlier. TIM190 was included as an example of an irreversible inhibitor. Plots were generated in Prism (GraphPad), and figure was assembled in PowerPoint.

FIG. 5: Inhibition Kinetics of Lead Compounds. Comparison of dose-response curves from IC₅₀ experiments for lead compounds using linearized plasmid (‘[S] < Km’) versus self-annealing 30-mer (‘[S] » Km’) substrate. The two substrates were used in standard reactions with equal mass quantities of DNA to facilitate inter-experiment comparisons of the fluorescence-based assay. Reactions with the 30-mer have [S]~1 µM, and those with the plasmid have [S]~1.7 nM. The compounds’ mechanism of inhibition was classified based on the relationship between these two IC₅₀. Panel A: A summary chart of the lead compounds’ inhibition kinetics. IC₅₀ ratios were calculated as the ‘[S] » Km’ IC₅₀ divided by the ‘[S] < Km’ IC₅₀. Compounds were classified by these ratios as follows: 0.05-0.2 → Uncompetitive, 0.5-2.0 → Noncompetitive, 30-120 → Competitive, Else → Mixed. TIM218 was a special case: the ratio was stated as >20 based on the upper concentration of inhibitor tested, but the trending dose-response curve suggests a ratio of ~50. Panels B-F: Overlays of the dose-response curves generated using the two different substrates. Plots were generated in Prism (GraphPad) as described earlier, and figure was assembled in PowerPoint.

FIG. 6: H-NMR Structural Validation of Lead Compounds. Panel A: 2D chemical structures of lead compounds, provided for reference. Structures were generated in ChemDraw (Perkin Elmer). Panels B-F: Proton NMR graphs for lead compounds. Singlet peaks at 2.2, 2.5, & 3.3 PPM may correspond to contaminating acetone, DMSO, and water, respectively.

FIG. 7: Validated Classification of TIM009 Mechanism of Inhibition. The most potent lead compound, TIM009, had its inhibition kinetics confirmed via a gel assay to validate the efficacy of the fluorescence-based assay. Standard reactions of 100 uL were prepared with the indicated concentrations of inhibitor and either 15 nM or 515 nM of FAM-labeled 30-mer oligo. Reactions are incubated at room temperature for 20 min, quenched, dried in vacuo, resuspended, and visualized on a urea-polyacrylamide gel. Initial velocities of each reaction are calculated via densitometric analysis of the gel, and then normalized to vehicle control reactions to calculate percent activities. Dose-response plots are fitted with nonlinear regression to interpolate IC₅₀. Classifications were made as described for FIG. 4, and they were in agreement with the fluorescence assay. Panel A: Gel for the ‘[S] < Km’ reactions. Reactions are shown in triplicate lanes, with inhibitor concentration increasing as indicated. ‘Ladder’ is a reaction with excessive enzyme to generate a clear banding pattern for reference during quantification. Panel B: Overlaid dose-response curves for TIM009. Plot was prepared in Prism (GraphPad).

### DETAILED DESCRIPTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

The disclosures of all patent references cited herein are hereby incorporated by reference to the extent they are consistent with the disclosure set forth herein. As used herein in the description of the invention and the appended claims, the singular forms “a,” “an” and “the” are intended to include the plural forms as well, unless the context clearly indicates otherwise.

The TREX1 protein and TREX1 gene (e.g., the “wild type” TREX1 protein and gene) are known and described in, for example, U.S. Pat. No. 6,632,665 to Fred W. Perrino (human and mouse), the disclosure of which is incorporated herein by reference in its entirety. The Mus musculus TREX1 sequence is also described at NCBI Reference Sequence: NP_035767.4.

As used herein in the accompanying chemical structures, “H” refers to a hydrogen atom. “C” refers to a carbon atom. “N” refers to a nitrogen atom. “S” refers to a sulfur atom. “O” refers to an oxygen atom.

“Alkyl,” as used herein, refers to a saturated straight or branched chain, or cyclic hydrocarbon containing from 1 to 10 carbon atoms (e.g. C₁₋₃alkyl, C₁₋₄alkyl, C₁₋₅alkyl, etc.). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, cyclopropyl, cyclobutyl, cyclopentyl, cyclohexyl, and the like. “Lower alkyl” as used herein, is a subset of alkyl and refers to a straight or branched chain hydrocarbon group containing from 1 to 4 carbon atoms. Representative examples of lower alkyl include, but are not limited to, methyl, ethyl, n-propyl, iso-propyl, n-butyl, iso-butyl, tert-butyl, cyclopropyl, cyclobutyl, and the like. The alkyl groups may be optionally substituted with one or more suitable substituents, such as halo, hydroxy, carboxy, amine, etc.

“Alkenyl,” as used herein, refers to a straight or branched chain hydrocarbon containing from 2 to 10 carbons and containing at least one carbon-carbon double bond formed by the removal of two hydrogens. Representative examples of “alkenyl” include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl and the like. “Lower alkenyl” as used herein, is a subset of alkenyl and refers to a straight or branched chain hydrocarbon group containing from 2 to 4 carbon atoms and at least one carbon-carbon double bond.

“Alkynyl,” as used herein, refers to a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, 1-butynyl and the like. “Lower alkynyl” as used herein, is a subset of alkynyl and refers to a straight or branched chain hydrocarbon group containing from 2 to 4 carbon atoms at least one carbon-carbon triple bond.

“Aryl,” as used herein, refers to a monocyclic carbocyclic ring system or a bicyclic carbocyclic fused or directly adjoining ring system having one or more aromatic rings. Examples include, but are not limited to, phenyl, indanyl, indenyl, tetrahydronaphthyl, and the like. As noted, in some embodiments, the aryl has two aromatic rings, which rings are fused or directly adjoining. Examples include, but are not limited to, biphenyl, napthyl, azulenyl, etc. The aryl may be optionally substituted with one or more suitable substituents, such as alkyl, halo, hydroxy, carboxy, amine, etc.

“Heteroaryl,” as used herein, refers to a monovalent aromatic group having a single ring or two fused or directly adjoining rings and containing in at least one of the rings at least one heteroatom (typically 1 to 3) independently selected from nitrogen, oxygen and sulfur. Examples include, but are not limited to, pyrrole, imidazole, thiazole, oxazole, furan, thiophene, triazole, pyrazole, isoxazole, isothiazole, pyridine, pyrazine, pyridazine, pyrimidine, triazine, and the like. As noted, in some embodiments, the heteroaryl has two aromatic rings, which rings are fused or directly adjoining. Examples include, but are not limited to, benzothiophene, benzofuran, indole, benzimidazole, benzothiazole, quinoline, isoquinoline, quinazoline, quinoxaline, phenyl-pyrrole, phenyl-thiophene, etc. The heteroaryl may be optionally substituted with one or more suitable substituents, such as alkyl, halo, hydroxy, carboxy, amine, etc.

The terms “halo” and “halogen,” as used herein, refer to fluoro (-F), choro (-Cl), bromo (-Br), or iodo (-I).

“Alkoxy,” as used herein, refers to an alkyl group, as defined herein, appended to the parent molecule through an oxygen atom (-O-). Representative examples of alkoxy include, but are not limited to, methoxy, ethoxy, propoxy, 2-propoxy, butoxy, tert-butoxy, pentyloxy, hexyloxy and the like.
“Carboxy” refers to the group —COOH.
“Sulfonate” refers to the group —SO₃⁻
“Hydroxyl” and “hydroxy” refer to the group —OH.
“Nitrile” refers to the group —CN.

As understood in the art, the term “optionally substituted” indicates that the specified group is either unsubstituted, or substituted by one or more suitable substituents. A “substituent” that is “substituted” is a group which takes the place of one or more hydrogen atoms on the parent organic molecule.

### I Active Compounds

Active compounds useful as TREX1 inhibitors in accordance with the present invention are provided below. Unless otherwise stated, structures depicted herein are also meant to include all enantiomeric, diastereomeric, and geometric (or conformational) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Tautomeric forms include keto-enol tautomers of a compound. In addition, unless otherwise stated, all rotamer forms of the compounds of the invention are within the scope of the invention. Unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

Provided herein according to some embodiments as an active compound is a compound of Formula I, a compound of Formula II, a compound of Formula III, a compound of Formula IV, or a compound of Formula V:

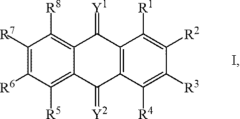

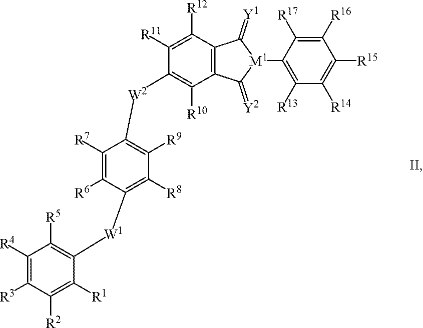

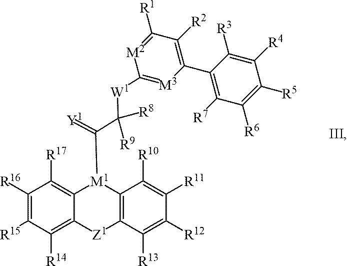

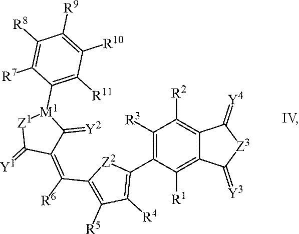

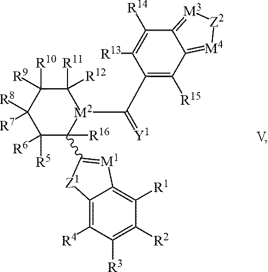

wherein:
M¹, M², M³, and M⁴ are each independently N or C-R′, wherein R′ is H, OH, C₁₋₅ alkyl, halogen, sulfonate, carboxy, C₁₋₅alkoxy, or nitrile;
W¹, W², Y¹, Y², Y³ and Y⁴ are each independently O or S;
Z¹, Z² and Z³ are each independently selected from O, S, N-R″, wherein R″ is H or optionally substituted alkyl, and CR^{a}R^{b}, wherein R^{a} and R^{b} are each independently selected from H, OH, alkoxy, C₁₋₅ alkyl, halogen, sulfonate, carboxy, C₁₋₅alkoxy, and nitrile; and
R¹ to R¹⁷ are each independently selected from H, OH, C₁₋₅ alkyl, halogen, sulfonate, carboxy, C₁₋₅alkoxy, and nitrile,
or a pharmaceutically acceptable salt or prodrug thereof.

In some embodiments, the compound is a compound of Formula I:

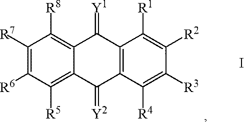

wherein:
Y¹ and Y² are each O;
R¹ and R² are each hydroxy;
R³ is sulfonate
R⁴ is H; and
R⁵, R⁶, R⁷, and R⁸ are each independently H or C₁₋₅alkyl,
or a pharmaceutically acceptable salt or prodrug thereof.

In some embodiments, the compound is a compound of Formula II:

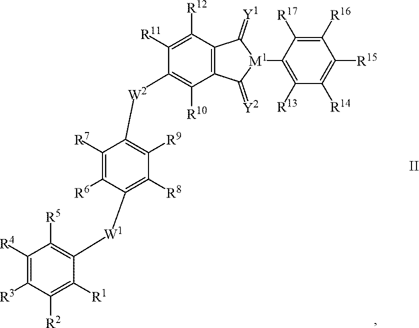

wherein:
Y¹ and Y² are each O;
W¹ and W² are each O;
M¹ is N;
one of R¹ to R⁵ is carboxy and the others are H;
R⁶ to R¹⁴, R¹⁶ and R¹⁷ are each independently H or C₁₋₅alkyl; and
R¹⁵ is alkoxy (e.g. methoxy),
or a pharmaceutically acceptable salt or prodrug thereof.

In some embodiments, the compound is a compound of Formula III:

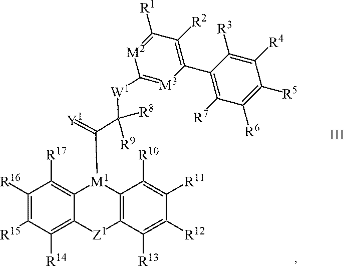

wherein:
Y¹ is O;
W¹ is S;
M¹ is N;
Z¹ is S;
M² and M³ are each N;
R¹ is hydroxyl;
R² is nitrile; and
R³ to R¹⁷ are each independently H or C₁₋₅alkyl,
or a pharmaceutically acceptable salt or prodrug thereof.

In some embodiments, the compound is a compound of Formula IV:

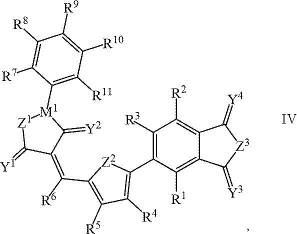

wherein:
Y¹, Y², Y³ and Y⁴ are each O;
Z¹ is NH;
M¹ is N;
Z² is O;
Z³ is NH;
R¹ to R⁸ and R¹¹ are each independently H or C₁₋₅alkyl; and
R⁹ and R¹⁰ are each C₁₋₅alkyl (e.g. methyl),
or a pharmaceutically acceptable salt or prodrug thereof.

In some embodiments, the compound is a compound of Formula V:

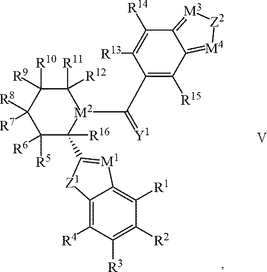

wherein:
Y¹ is O;
Z¹ is NH;
M¹ is N;
M², M³, and M⁴ are each N;
Z² is O;
one of R¹ to R⁴ is halo (e.g. fluoro) and the others are H; and
R⁵ to R¹⁶ are each independently H or C₁₋₅alkyl,
or a pharmaceutically acceptable salt or prodrug thereof.

Particular examples of active compounds in accordance with the present invention are as follows:

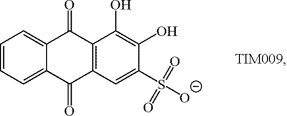 [SMILES: O=C1c2ccccc2C(=O)c2c1cc(S(=O)(=O)[O-])c(O)c2O]

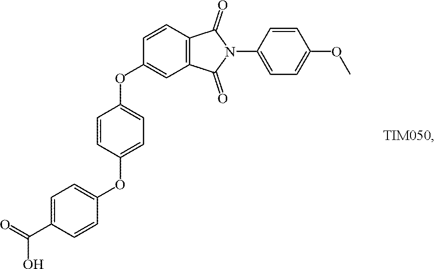 [SMILES: COc1ccc(N2C(=O)c3ccc(Oc4ccc(Oc5ccc(C(=O)O)cc5)cc4)cc3C2=O)cc1]

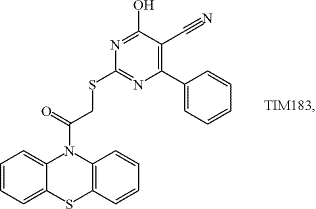 [SMILES: N#Cc1c(O)nc(SCC(=O)N2c3ccccc3Sc3ccccc32)nc1-c1ccccc1]

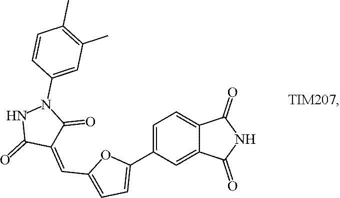 [SMILES: Cc1ccc(N2NC(=O)/C(=C/c3ccc(-c4ccc5c(c4)C(=O)NC5=O)o3)C2=O)cc1C]

and

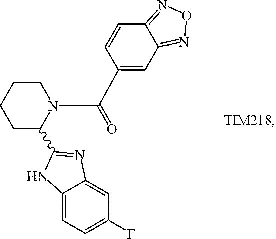 [SMILES: O=C(c1ccc2nonc2c1)N1CCCCC1c1nc2cc(F)ccc2[nH]1]

inclusive of pharmaceutically acceptable salts thereof.

Also provided as an active compound in accordance with the present invention is a compound of Formula VI, a compound of Formula VII, a compound of Formula VIII, or a compound of Formula IX:

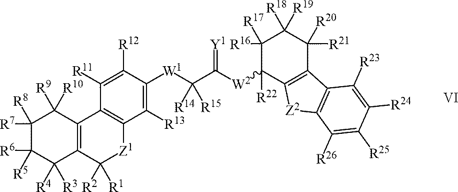

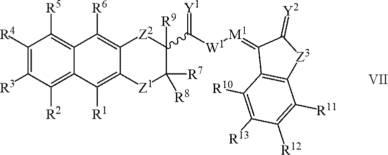

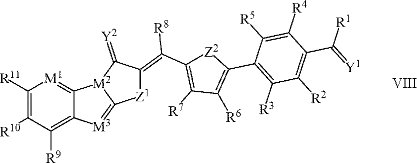

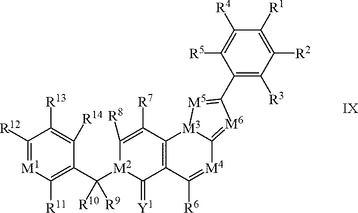

wherein:
M¹ to M⁶ are each independently N or C-R′, wherein R′ is H, OH, C₁₋₅ alkyl, halogen, sulfonate, carboxy, C₁₋₅alkoxy, or nitrile;
W¹, W², Y¹ and Y² are each independently O or S;
Z¹, Z², an Z³ are each independently selected from O, S, N-R″, wherein R″ is H or optionally substituted alkyl, and CR^{a}R^{b}, wherein R^{a} and R^{b} are each independently selected from H, OH, alkoxy, C₁₋₅ alkyl, halogen, sulfonate, carboxy, C₁₋₅alkoxy, and nitrile; and
R¹ to R²⁶ are each independently selected from H, OH, C₁₋₅ alkyl, halogen, sulfonate, carboxy, C₁₋₅alkoxy, and nitrile,
or a pharmaceutically acceptable salt or prodrug thereof.

In some embodiments, the compound is a compound of Formula VI:

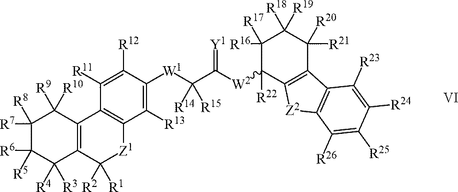

wherein:
Y¹ is O;
Z¹ is O;
Z² is NH;
W¹ is O;
W² is NH;
one of R²³ to R²⁶ is C₁₋₅alkoxy (e.g., methoxy) and the others are each H; and
R¹ to R²² are each H,
or a pharmaceutically acceptable salt or prodrug thereof.

In some embodiments, the compound is a compound of Formula VII:

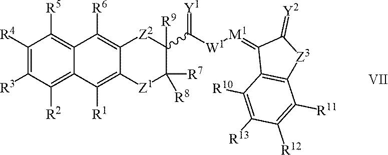

wherein:
W¹ is NH;
Y¹ and Y² are each O;
Z¹ and Z² are each O;
Z³ is NR″, wherein R″ is lower alkyl (e.g. methyl);
M¹ is N;
R¹ to R⁹ are each H; and
one of R¹⁰ to R¹³ is halo (e.g. bromo) and the others are each independently H or lower alkyl,
or a pharmaceutically acceptable salt or prodrug thereof.

In some embodiments, the compound is a compound of Formula VIII:

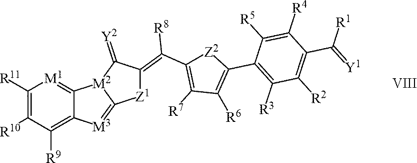

wherein:
Y¹ and Y² are each O;
Z¹ is S;
Z² is O;
M¹, M², and M³ are each N;
R¹ is OH;
one of R⁹ to R¹¹ is halo (e.g. bromo), and the others are H; and
R² to R⁸ are each H,
or a pharmaceutically acceptable salt or prodrug thereof.

In some embodiments, the compound is a compound of Formula IX:

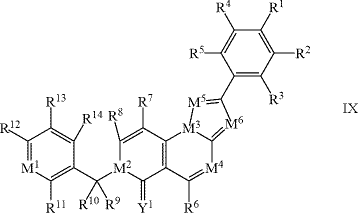

wherein:
Y¹ is O;
M¹ to M⁶ are each N;
one of R¹ to R⁵ is halo (e.g. chloro), and the others are H; and
R⁶ to R¹⁴ are each H,
or a pharmaceutically acceptable salt or prodrug thereof.

Particular examples of active compounds include:

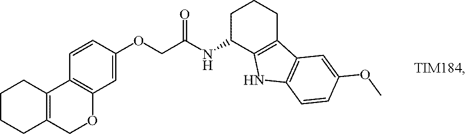 [SMILES: COc1ccc2[nH]c3c(c2c1)CCC[C@H]3NC(=O)COc1ccc2c(c1)OCC1=C2CCCC1]

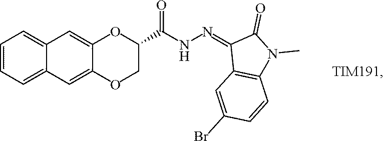 [SMILES: CN1C(=O)/C(=N/NC(=O)[C@@H]2COc3cc4ccccc4cc3O2)c2cc(Br)ccc21]

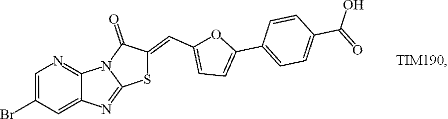 [SMILES: O=C(O)c1ccc(-c2ccc(/C=c3\sc4nc5cc(Br)cnc5n4c3=O)o2)cc1]

and

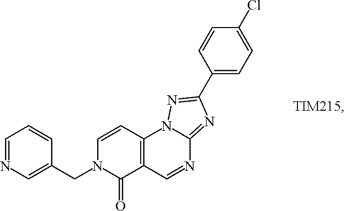 [SMILES: O=c1c2cnc3nc(-c4ccc(Cl)cc4)nn3c2ccn1Cc1cccnc1]

and pharmaceutically acceptable salts thereof.

### II. Methods of Use

Modulating the innate immune system through TREX1 and/or the cGAS-STING pathway has the potential to address a variety of diseases. For example, enhancement of the innate immune response can help treat viral infections and cancers. See also US 2020/0224200 to Sharma et al.

Activation of the innate immune response in a controlled manner may also help mitigate the effects of autoimmune flare ups by holding the immune response in an idling position. Thus, active compounds taught herein that modulate the innate immune system may be used in a method of treating a cancer, viral infection, or autoimmune disease.

The terms “treat”, “treatment” and “treating” as used herein refer to any type of treatment that imparts a benefit to a subject afflicted with or at risk of an injury, infection, disease or disorder, delay in the progression of the injury, infection, disease or disorder, or symptoms thereof, etc.

In some embodiments, the subject is a human subject. In some embodiments, the subject is a non-human animal (e.g., non-human mammalian subject). A non-human animal may include, but is not limited to, non-human primates, dogs, cats, horses, cattle, goats, pigs, sheep, guinea pigs, mice, rats and rabbits, as well as any other domestic, commercially or clinically valuable animal, including but not limited to animal models and livestock animals.

Enhancement of the immune response in the treatment of cancer is known. Such cancers may include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bile duct cancer; bladder cancer; bone cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cardiac tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ductal carcinoma in situ; ependymoma; endotheliosarcoma (e.g., Kaposi’s sarcoma, multiple idiopathic hemorrhagic sarcoma); endometrial cancer (e.g., uterine cancer, uterine sarcoma); esophageal cancer (e.g., adenocarcinoma of the esophagus, Barrett’s adenocarcinoma); Ewing’s sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom’s macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; multiple myeloma; heavy chain disease (e.g., alpha chain disease, gamma chain disease, mu chain disease); hemangioblastoma; histiocytosis; hypopharynx cancer; inflammatory myofibroblastic tumors; immunocytic amyloidosis; kidney cancer (e.g., nephroblastoma a.k.a. Wilms’ tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); melanoma; midline tract carcinoma; multiple endocrine neoplasia syndrome; muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); nasopharynx cancer; neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); parathryroid cancer; papillary adenocarcinoma; penile cancer (e.g., Paget’s disease of the penis and scrotum); pharyngeal cancer; pinealoma; pituitary cancer; pleuropulmonary blastoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; retinoblastoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; stomach cancer; small intestine cancer; sweat gland carcinoma; synovioma; testicular cancer (e.g., seminoma, testicular embryonal carcinoma); thymic cancer; thyroid cancer (e.g., papillary carcinoma of the thyroid, papillary thyroid carcinoma (PTC), medullary thyroid cancer); urethral cancer; uterine cancer; vaginal cancer; and vulvar cancer (e.g., Paget’s disease of the vulva). See also US 2019/0153098 to Goldberg et al.

In some embodiments, the cancer is a blood cancer such as leukemia, liver cancer, lung cancer, lymphoma, melanoma, bladder cancer, brain cancer, breast cancer, or cervical cancer.

Enhancement of the immune response in the treatment of a viral infection is known. The viral infection may include, but is not limited to, an infection of Hepatitis A, Hepatitis B, Hepatitis C, Hepatitis D, Human papillomavirus, Human T-cell lymphotropic virus, Coronaviruses, Rubella, Mumps, Coxsackie virus A, Coxsackie virus B, Human enteroviruses, Poliovirus, Viral encephalitides viruses, Human herpesviruses including Cytomegalovirus, Epstein-Barr viruses, Human herpesviruses, Herpes B virus, Herpes simplex viruses, Varicella zoster virus, Human immunodeficiency viruses, Reoviruses, Rhinoviruses, Adenoviruses, Filoviruses, including Marburg virus and Ebola virus, Arenaviruses including lymphocytic choriomeningitis virus, Lassa virus, Junin virus, and Machupo Virus, Rabies virus, Arboviruses including West Nile virus, Dengue viruses, Colorado tick fever virus, Sinbis virus, Togaviraidae, Flaviviridae, Bunyaviridae, Reoviridae, and Rhabdoviridae, Poxviruses, Yellow fever virus, Hantaviruses, Measles virus, Human parainfluenza viruses, Influenza viruses, Respiratory syncytial viruses, Rotaviruses, Polyomaviruses, Coltiviruses, Calciviruses, and Parvoviruses. See also US 2020/0131209 to Diller et al.

An autoimmune disease that may be treated with a compound as described herein includes, but is not limited to, systemic lupus erythematosus (SLE); Aicardi-Goutieres syndrome (AGS); familial chilblain lupus (FCL); STING-associated vaculopathy with onset infancy (SAVI), Sjögren’s syndrome; schleroderma; and retinal vasculopathy with cerebral leukodystrophy (RVCL), etc.

### III. Formulations

The compounds as taught herein may be provided in a salt form such as a pharmaceutically acceptable salt. A “pharmaceutically acceptable salt” is a salt that retains the biological effectiveness of the free acids and bases of a specified compound and that is not biologically or otherwise undesirable. Examples of pharmaceutically acceptable salts include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycollates, tartrates, methane-sulfonates, propanesulfonates, naphthalene-1-sulfonates, naphthalene-2-sulfonates, and mandelates.

“Prodrug” as used herein is a drug or pharmaceutical agent that is inactive in its administered form, but becomes a pharmacologically active agent by a metabolic or physicochemical transformation. A thorough discussion is provided in T. Higuchi and V. Stella, Prodrugs as Novel delivery Systems, Vol. 14 of the A.C.S. Symposium Series and in Edward B. Roche, ed., Bioreversible Carriers in Drug Design, American Pharmaceutical Association and Pergamon Press, 1987, both of which are incorporated by reference herein in their entireties. See also US 2020/0010432 to Huigens, III, et al., U.S. Pat. Nos. 5,696,126 and 6,680,299, and “Prodrug Design of Phenolic Drugs,” DOI: 10.2174/138161210791293042, herein incorporated by reference.

The active compounds described herein may be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the active compound (including the physiologically acceptable salts or prodrugs thereof) is typically admixed with, inter alia, an acceptable carrier. The carrier must, of course, be acceptable in the sense of being compatible with any other ingredients in the formulation and must not be deleterious to the subject. The carrier may be a solid or a liquid, or both, and is preferably formulated with the compound as a unit-dose formulation, for example, a tablet, which may contain from 0.01 or 0.5% to 95% or 99% by weight of the active compound. One or more active compounds may be incorporated in the formulations of the invention, which may be prepared by any of the well-known techniques of pharmacy comprising admixing the components, optionally including one or more accessory ingredients.

The pharmaceutical compositions may also contain other additives, such as pH-adjusting additives. In particular, useful pH-adjusting agents include acids, such as hydrochloric acid, bases and/or buffers, such as sodium lactate, sodium acetate, sodium phosphate, sodium citrate, sodium borate, or sodium gluconate. Further, the compositions may contain preservatives. Useful preservatives include methylparaben, propylparaben, benzoic acid and benzyl alcohol.

Formulations of the invention include those suitable for oral, buccal (sub-lingual), parenteral (e.g., subcutaneous, intramuscular, intradermal, or intravenous), topical (i.e., both skin and mucosal surfaces, including airway surfaces) and transdermal administration, although the most suitable route in any given case will depend on the nature and severity of the condition being treated and on the nature of the particular active compound being used.

Formulations suitable for oral administration may be presented in discrete units, such as capsules, cachets, lozenges, or tablets, each containing a predetermined amount of the active compound(s); as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for oral administration also include food product formulations, such as a nutritional bar or an animal feed (e.g., pet food such as dog or cat food). Food product formulations may include one or more of carbohydrates such as wheat, corn rice, barley or oats, dairy products such as milk, oils such as canola oil or soybean oil, flavorants such as sugar or syrup, coloring, chocolate, preservatives, etc. Pet food formulations, in particular, may include meat, poultry, fish or other animal-derived components such as eggs.

Formulations suitable for buccal (sub-lingual) administration include lozenges comprising the active compound in a flavored base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions, which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising an active compound(s) in a unit dosage form in a sealed container. The active compound(s) may be provided in the form of a lyophilizate which is capable of being reconstituted with a suitable pharmaceutically acceptable carrier to form a liquid composition suitable for injection thereof into a subject.

When the active compound(s) is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound. Suitable formulations comprise citrate or bis\tris buffer (pH 6) or ethanol/water and contain from 0.1 to 0.2 M active ingredient.

The amount of active compound(s) administered for therapeutic treatment may depend on the age, weight, and condition of the patient as determined by a physician. The unit dosage form typically comprises from about 1 mg, 5 mg, 10 mg, 100 mg, 250 mg, 500 mg, 1 gram, 5 grams, 10 grams, or any ranges therein, of the active compound(s), depending on the subject being treated (e.g., human or non-human mammalian subject). In some embodiments, the unit dosage form is in the range of 500 mg to 10 grams, keeping in mind that a good portion of the active compound(s) may not be absorbed upon administration (e.g., oral adminstration).

In some embodiments, the present invention provides a pharmaceutical composition which is utilized in combination with radiation therapy.

The present invention is explained in greater detail in the following non-limiting examples.

### EXAMPLES

### Example 1: Library Selection, Virtual Screening, & High-Throughput Screening (HTS)

We applied structure-based virtual screening (SBVS) to the commercial 150k-compound ChemDiv Diversity Library and the ~1.2M-compound ChemBridge CORE+EXPRESS libraries. Compounds were docked to a single active-site of a mTREX1-dAMP crystal structure (PDB= ‘2IOC’), which had the dAMP atoms removed in PyMol (Schrodinger), using AutoDock Vina⁵⁹-⁶¹. A murine TREX1 crystal structure was used, since no crystal structures of the human enzyme exist. The top-scoring 698 and 2004 compounds from the ChemDiv and ChemBridge libraries, respectively, were selected for further study and ordered as physical libraries. In addition to these libraries, we also had access to an in-house library of 8800 compounds (Nanosyn, Inc) purchased by other labs at our institution.

We altered a reported fluorescence-based exonuclease assay^{17,23} for the purposes of HTS. The assay parameters were optimized to limit materials use and maximize the dynamic range of the assay readout. We used this assay to perform a HTS of the three mentioned libraries against recombinant hTREX1(1-242)^{WT}, hereafter abbreviated as ‘hT1’. In the case of all libraries, compounds eliciting ≤50% activity at their indicated screening concentrations were identified as ‘candidates’.

All ChemDiv and Chembridge candidates plus 51/222 (~23%) of the Nanosyn candidates were commercially available, giving a combined total of 112 candidates for further testing. The compounds are available from one or more of the following vendors: Ambinter, ChemDiv, ChemBridge, Nanosyn, Sigma-Aldrich.

### Example 2: Determining IC₅₀ & Chemotypes

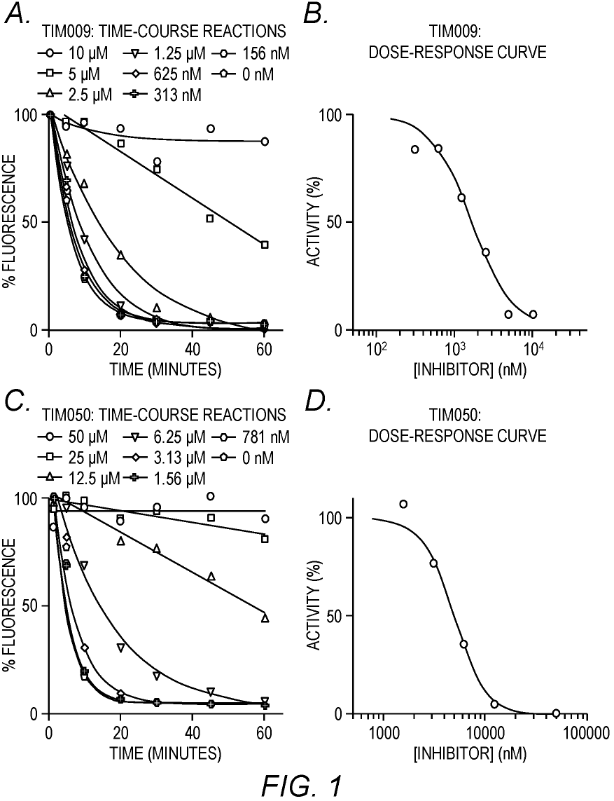

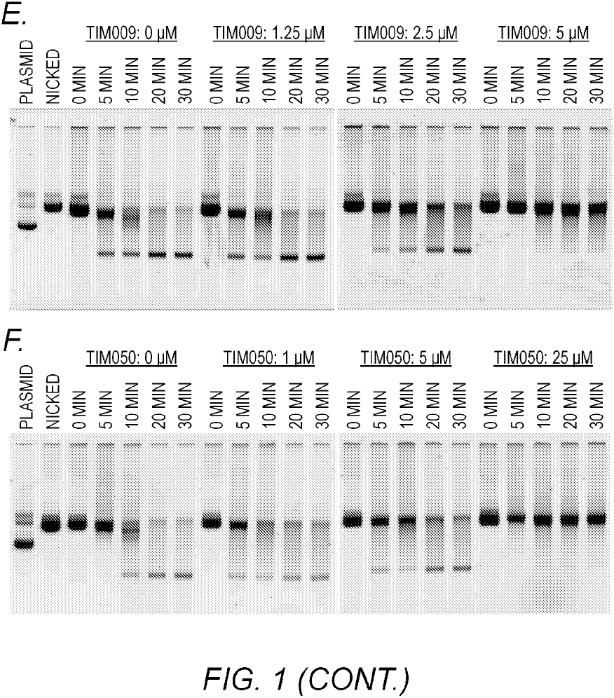

The biochemical assay used for HTS was also applied to determining candidate IC₅₀. Briefly, time-course reactions were performed for a range of concentrations for each candidate, and plots of fluorescence vs time were normalized to the initial fluorescence of the reaction and the plate-wide background fluorescence. This normalization helped address compounds with inherent effects on the fluorescent readout. Initial velocities were calculated from normalized plots and used to calculate percent activities relative to vehicle-only control reactions. Standard dose-response curves were then constructed to interpolate candidate IC₅₀. Example data for the two most potent candidates is provided (FIG. 1, Panels A-D). Candidates that initially tested with IC₅₀ ≤100 µM had their IC₅₀ redetermined in two additional, independent experiments. Of the 112 candidates, 73 (65%) had a median IC₅₀ ≤100 µM and were identified as ‘actives’, and 20 (18%) had a median IC₅₀ ≤15 µM and were identified as ‘hits’. The remaining candidates had IC₅₀ >100 µM or were completely inactive. To validate the efficacy of the fluorescence-based assay at determining IC₅₀, several of the hit compounds had their IC₅₀ confirmed using a gel assay previously described by our lab²³. Briefly, a dsDNA plasmid is nicked, the substrate is incubated with TREX1 which degrades off (partially or completely) the nicked strand, and the reaction is quenched in ethanol, dried in vacuo, and visualized via agarose gel electrophoresis. We found that results of the gel assays confirmed the IC₅₀ of our tested hit compounds, which are presented in TABLE 1. Example data of these validation assays is provided (FIG. 1, Panels E-F).

TABLE 1

HIT COMPOUND IC50 DATA

| Compound | PubChem CID | Median IC50 (µM) | IC50 #1 (µM) | IC50 #2 (μΜ) | IC50 #3 (μΜ) |
|---|---|---|---|---|---|
| TIM009 | 3863551 | 1.1 | 0.3 | 1.1 | 1.5 |
| TIM050 | 3538689 | 3.7 | 3.7 | 3.0 | 5.3 |
| TIM181 | 46102878 | 4.6 | 3.5 | 5.9 | 4.6 |
| TIM213 | 2286239 | 5.0 | 2.4 | 5.0 | 10.7 |
| TIM137 | 40472664 | 6.7 | 6.7 | 16.2 | 5.6 |
| TIM178 | 50832545 | 6.9 | 6.9 | 3.7 | 7.2 |
| TIM184 | 97489970 | 7.0 | 5.1 | 7.0 | 7.0 |
| TIM148 | 4099157 | 8.5 | 6.4 | 8.5 | 11.9 |
| TIM183 | 135520808 | 8.5 | 3.6 | 8.5 | 8.8 |
| TIM191 | 1364406 | 8.5 | 8.5 | 7.8 | 10.0 |
| TIM003 | 1338201 | 8.8 | 24.9 | 7.8 | 8.8 |
| TIM207 | 2276687 | 8.9 | 13.5 | 7.8 | 8.9 |
| TIM136 | 1364790 | 9.9 | 5.7 | 9.9 | 10.2 |
| TIM190 | 10162140 | 10.7 | 10.7 | 10.7 | 13.5 |
| TIM218 | 95897263 | 12.7 | 12.7 | 7.6 | 17.8 |
| TIM192 | 2844785 | 13.0 | 13.3 | 13.0 | 8.1 |
| TIM215 | 16653542 | 13.9 | 13.9 | 14.6 | 12.0 |
| TIM185 | 41033057 | 14.9 | 13.1 | 14.9 | 113.0 |
| TIM146 | 2398456 | 15.0 | 15.0 | 6.4 | 37.9 |
| TIM047 | 6151830 | 15.0 | 16.0 | 6.8 | 15.0 |

The relationship between a compound’s structure and some measured activity is referred to as structure-activity relationship (SAR) and is a key consideration of modern drug discovery. Thus, several methods to classify small molecules into structurally related groupings (‘chemotypes’) have been proposed⁶⁵⁻⁶⁷. We employed a previously described hierarchal chemotyping strategy⁶⁵, using the software Strip-it (Silicos-it)⁶⁸. With this strategy, compounds with identical cyclic system skeletons (CSS) are classified as the same chemotype, and compounds therein with identical cyclic systems (CS) are considered most closely related. Using this strategy, we grouped our active compounds into chemotypes. We found 7 chemotypes that each contained two actives, 1 chemotype that contained 4 actives, and 55 of the actives belonged to a unique chemotype. We also sought to elucidate if our inhibitors may have any common pharmacophore contacts with TREX1. Thus, we determined the pharmacophore points that were most enriched among the docking poses of our hit compounds.

We found that there were four pharmacophore clusters, two hydrogen bond acceptors and two aromatic/lipophilic groups, that are represented among at least half our hits’ docking poses. Enrichment of the latter groups are unsurprising given the chemical features of the chemotypes identified, which possess multiple aromatic rings. Additionally, the two areas of hydrogen bond acceptor enrichment implicate the backbones of the L179, G180, and H195 residues as potential hydrogen bond donors in TREX1-inhibitor interactions. Overall, these data represent a potential insight into mechanisms of TREX1 inhibition.

### Example 3: Identifying Lead Compounds From Hits

To identify potential aggregators, we redetermined the IC₅₀ of hit compounds in the presence and absence of 0.01% TRITON™ X-100, and compared IC₅₀ between the two conditions. Reportedly, aggregators are detergent-sensitive, and should experience a drop in potency in the presence of detergent. We found 11/20 (55%) of our hit compounds experienced a 4-fold or greater reduction in potency in the presence of detergent, and we identified them as aggregators. For concentrations of TRITON™ X-100 below its critical micelle concentration of ~0.03% (CMC), we found no other explanation for this behavior in the literature.

Another common strategy for assessing the promiscuity of hit compounds is to employ counter-screens of the compounds against a protein unrelated to your target^{63,64}. For this purpose, we utilized recombinant wild-type Entamoeba histolytica thioredoxin reductase (EhTRXR) ⁷¹. We counter-screened our hit-compounds using the modified assay, and found that all of the aggregators either displayed significant assay-interference or discernable cross-activity. Of the nine detergent-insensitive compounds, we found that four similarly displayed assay-interference or cross-activity, suggesting possible non-specific action. The five hit compounds that were decidedly inactive against EhTRXR were identified as ‘leads’.

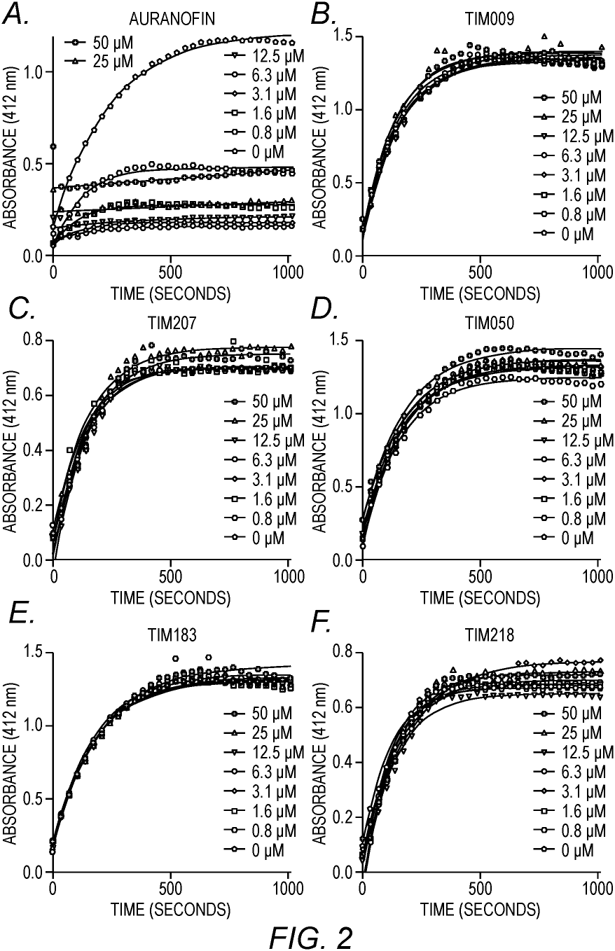

We were also interested in the activity of our leads against enzymes more closely related to hT1, namely murine TREX1 and the highly related human TREX2 enzyme. mTREX1 was of interest, since future studies of the lead compounds would presumably take place in a murine model. hTREX2, which also functions as a 3′➔5′ exonuclease, is primarily expressed in keratinocytes (while TREX1 expression is more ubiquitous), and its mutation is not associated with an autoimmune phenotype, but instead results in a hypermutator phenotype in the skin that is conditional upon exposure to ultraviolet radiation^{10,72-74}. For mTREX1, cross-activity was preferred to allow later efficacy studies in a murine model. While inactivity against hTREX2 is preferred, TREX2 mutant mice display no remarkable phenotype in the absence of UV challenge⁷³. However, given the striking similarity between these three enzymes, complete cross-activity of leads was a likelihood. We determined the IC₅₀ of lead compounds for recombinant wild-type murine TREX1(1-242), hereafter ‘mT1’, and for recombinant wild-type human TREX2(1-236), hereafter ‘hT2’ (FIG. 2). The same exonuclease assay described earlier was used for all enzymes. As expected, ⅘ leads were equally active against all three enzymes. However, the lead compound TIM218 imposed no detectable inhibition on mT1 or hT2 up to an inhibitor concentration of 100 µM. However, TIM218 did inhibit hT1. These results were consistent across multiple independent experiments, and the reason for this discrepancy in potencies is not yet clear.

For all leads we computed physicochemical properties reported to have strong correlations to drug-likeness and ADMET properties⁷⁵, including molecular weight (MW), number of hydrogen bond donors (HBD) and acceptors (HBA), topological polar surface area (TPSA), logarithm of partition coefficient (cLogP), number of rotatable bonds (RB), and logarithm of predicted solubility (cLogS). These values and the 2D chemical structures of the leads are provided in Table 2.

TABLE 2

Physicochemical Properties of Lead Compounds

| Identifier: | TIM009 | TIM050 | TIM183 | TIM207 | TIM218 |
|---|---|---|---|---|---|
| PubChem CID: | 3863551 | 3538689 | 135520808 | 2276687 | 95897263 |
| MW (Da): | 319.27 | 481.46 | 468.56 | 427.42 | 365.37 |
| clogP: | 0.78 | 5.78 | 5.64 | 2.91 | 3.61 |
| RB: | 1 | 7 | 4 | 3 | 2 |
| HBA: | 7 | 6 | 7 | 5 | 5 |
| HBD: | 2 | 1 | 1 | 2 | 1 |
| TPSA: | 132 | 102 | 90 | 109 | 88 |
| clogS (log[M]): | -2.73 | -2.98 | -4.77 | -4.60 | -2.93 |

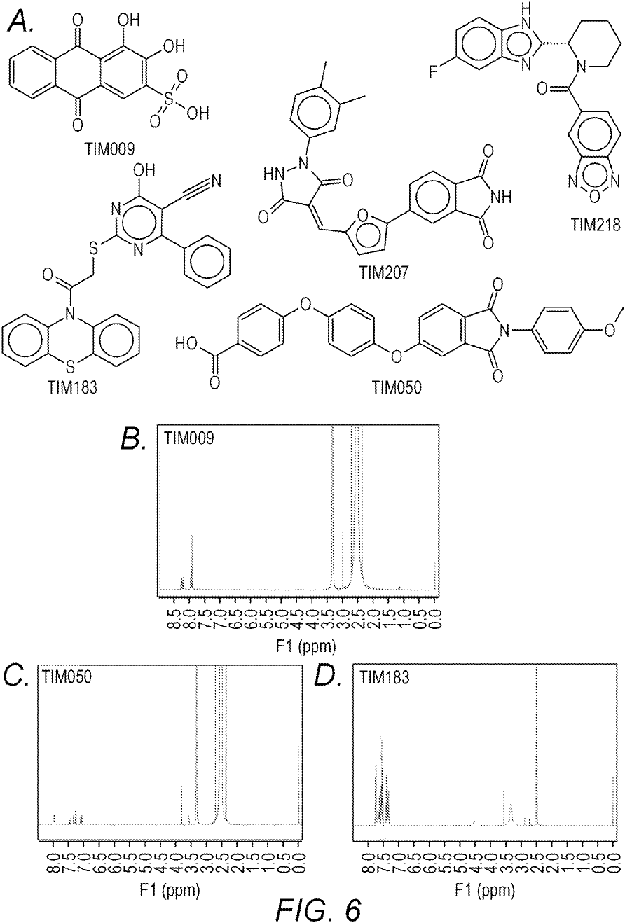

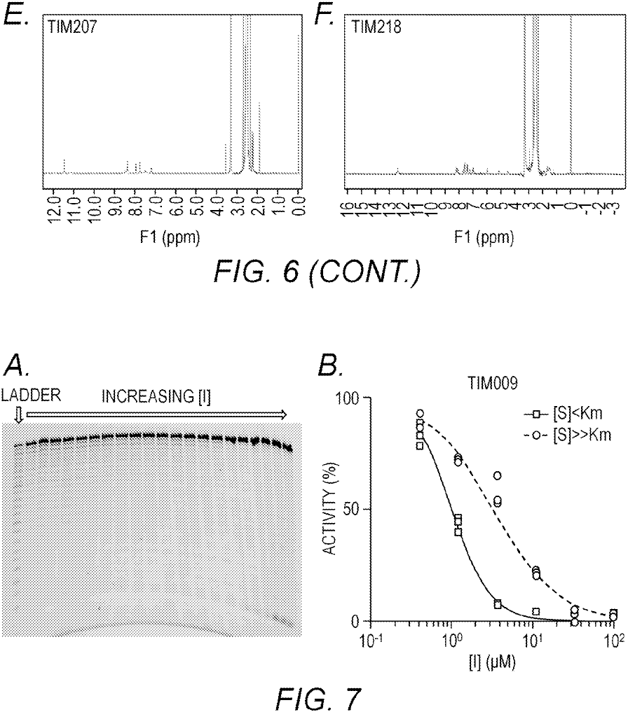

In addition, the leads’ chemical structures were validated by proton nuclear magnetic resonance (H-NMR), and these spectra are provided in FIG. 6. Minor violations of the cLogP parameters are noted for TIM050 and TIM183, but all leads adhere to Lipinski’s Rule of Fives (RO5)⁷⁵.

### Example 4: Classifying Leads’ Mechanism(s) of Inhibition (MOI)

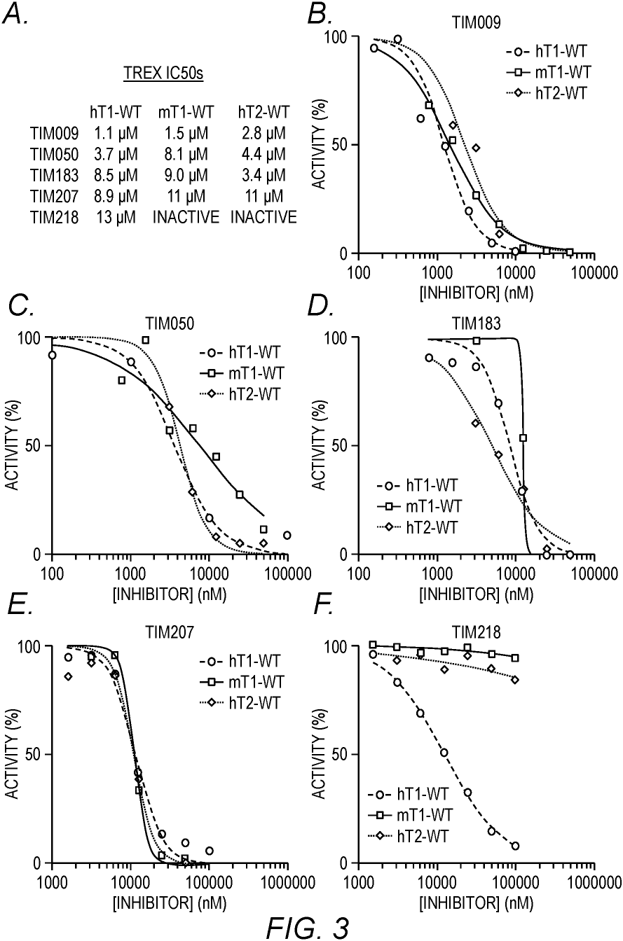

To determine whether our leads’ inhibition mechanisms were reversible, we utilized a strategy known as a ‘jump-dilution′⁷⁶. With this strategy, an inhibitor is initially incubated with the target at a high concentration, then, the inhibitor and target are diluted into reaction mix and the potency of the inhibitor is compared to a similar reaction without the pre-incubation step. If an inhibitor is covalent, time-dependent (slow-binding), or otherwise irreversible, then it will display potency consistent with the higher initial concentration, while reversible inhibitors’ potency is not meaningfully affected by the pre-incubation. We performed jump dilution experiments for our leads with our earlier described biochemical assay (FIG. 3). Under our conditions, irreversible inhibitors were expected to display a 10-fold potency increase in the jump-dilution reactions. Among our leads, we found that ⅘ had jump-dilution IC₅₀ within 2-fold that of the control reactions’, and were classified as ‘reversible’ (FIG. 3, Panel A). However, TIM207 exhibited a 2.9-fold increase in potency as a result of the jump-dilution, which we classified as ‘ambiguous’. A non-lead, TIM190, was also provided as an example of a compound whose potency was significantly affected by the jump-dilution, and consequently classified as ‘irreversible’ (FIG. 3, Panel B).

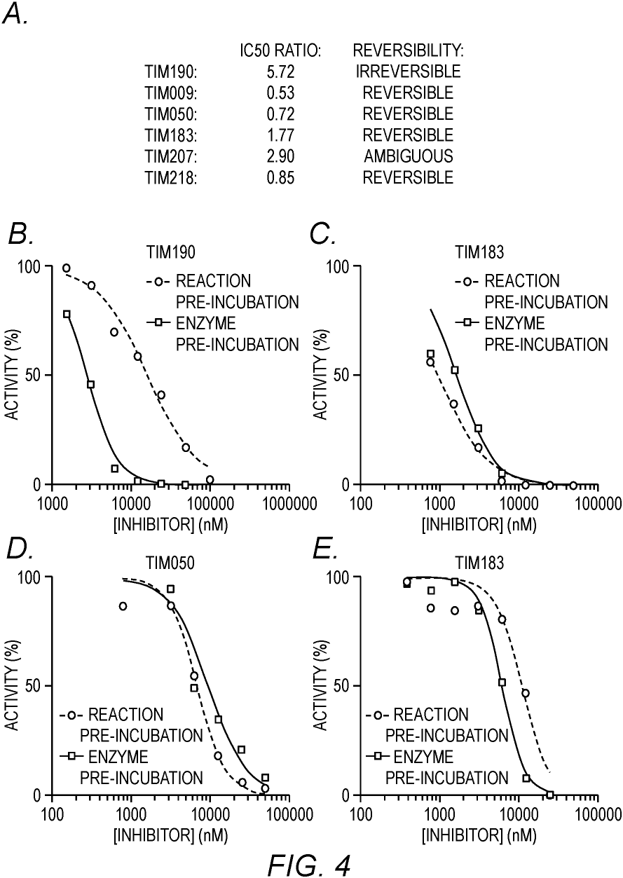

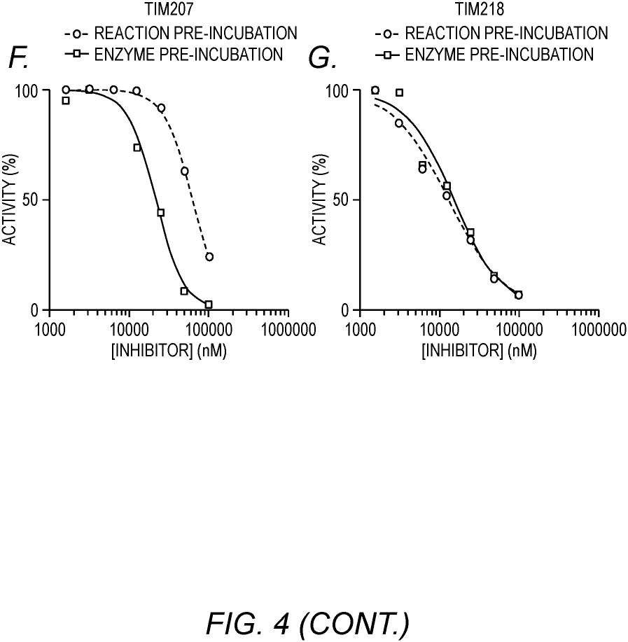

We also sought to characterize our leads’ MOI via standard inhibition kinetic definitions of competitive, noncompetitive, uncompetitive, or mixed⁷⁶⁻⁷⁸. The IC₅₀ of an inhibitor can be mathematically related to its inhibition constant (Kᵢ) and other kinetic parameters of the target, and the relationship varies with the MOI classification⁷⁹. Using these equations, we can simply determine the IC₅₀ of an inhibitor at substrate concentrations, [S], above and below the target Kₘ, then identify the MOI class that accurately predicts the ratio of the two IC₅₀. We determined these respective IC₅₀ for our lead compounds using a modified version of the biochemical assay described earlier (FIG. 4). In essence, the linearized dsDNA plasmid substrate was replaced with a self-annealing 30-mer oligonucleotide. Consequently, the concentration of three-prime hydroxyls in the reaction (the relevant measure of [S] for hT1) is increased from 1.7 nM ➔ 1000 nM. This substrate replacement keeps total mass of DNA in the assay constant and mimics the termini of the original substrate. Using our derived equations, these differential values for [S], and the previously reported constants for hT1¹⁰, the theoretical ratios for noncompetitive, competitive, and uncompetitive inhibitors in our assay were 1, ~60, and ~0.1, respectively. Leads with ratios that deviated from all theoretical values by more than 2-fold were classified as ‘mixed’ inhibitors.

We found that two of our leads were noncompetitive inhibitors, two were mixed inhibitors, and one was likely a competitive inhibitor (FIG. 4, Panel A). Even in the case of the two mixed inhibitors, their ratios were very close to being classified as noncompetitive. As a result, they presumably have similar affinities for the free enzyme (E) and enzyme-substrate complex (ES). To validate the efficacy of our fluorescence assay for this purpose, we also performed a similar analysis on the most potent lead, TIM009, using a previously described gel-based ssDNA degradation assay (FIG. 7)²³. Using this assay, we calculated a nearly identical IC₅₀ ratio for TIM009, and similarly identified it as a mixed inhibitor.

As a final assessment of the leads’ MOI, we performed exhaustive docking studies to the entirety of the mT1-apoenzyme homodimer structure. As with SBVS, a murine structure was used in the absence of existing human structures. We note similar docking scores for all leads, with the exception of a relatively low (more negative ΔG) score for TIM207 (Table 3).

TABLE 3

Lead Compound Docking Scores

| | ΔG (kcal/mol): |
|---|---|
| TIM009: | -9.1 |
| TIM050: | -9.1 |
| TIM183: | -9.2 |
| TIM207: | -10.1 |
| TIM218: | -9.1 |

This is not surprising, as TIM183 was discovered from one of the libraries prioritized by virtual screening. We also note that TIM218, which does not inhibit mT1 experimentally, has similar binding affinities to three of the four other inhibitors that did inhibit the mT1 enzyme. It is unclear from docking data alone how this result may be explained. It is possible that TIM218 binds as indicated but does not make contacts necessary for inhibition of mT1. Alternatively, the indicated docking pose may not be representative of TIM218′s binding mechanism, if it binds at all. Either way, the stark contrast in TIM218′s activity towards hT1 vs mT1 indicates structural differences in the enzymes are responsible. As gross differences in the mT1 and hT1 proteins’ backbone structures is unlikely, specific mT1-hT1 residue differences are implicated in TIM218′s binding mechanism. Furthermore, since the kinetic and docking data for TIM218 suggest it binds in or around the active site of TREX1, the L17, L19, S26, G80, A157, Q190, T192, and T203 residues are priority suspects for hT1-TIM218 contacts.

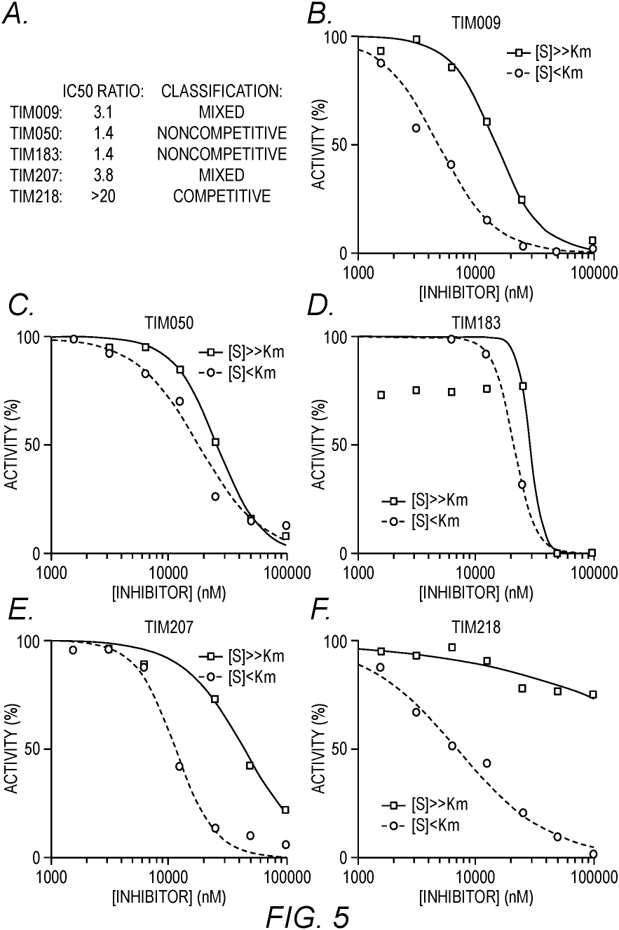

Interestingly, despite the variety of kinetic MOI classifications among our leads, all docked to the active site of either protomer (FIG. 5, Panels B-F). Though a dogmatic interpretation of the leads’ kinetic classifications implies that TREX1 can be bound to ⅘ of these inhibitors (all except TIM218) at the same time as substrate, the docking studies suggest that these inhibitors occupy a site on the enzyme where they would notably clash with bound substrate.

Given this, we reperformed the docking studies using a crystal structure of mT1-ssDNA to determine if comparable affinities existed for the E and ES structures. We found that the presence of ssDNA in the crystal structure shifted docking poses for all leads to the dimer interface of the enzyme, a known source of docking false-hits for dimeric enzymes. These pose locations, in addition to the notably increased docking scores (less negative ΔG) of each lead (Table 4), are in conflict with the kinetic data’s implication that the four relevant inhibitors have comparable affinities for E and ES. Therefore, to reconcile the biochemical and docking data we posit that these four leads must bind to a conformation of E and/or ES distinct from that captured in prior TREX1 crystal structures. To probe these hypotheses further, new TREX1-inhibitor crystal structures or robust molecular dynamics studies are warranted.

TABLE 4

Lead Compound Docking Scores

| | ΔG (kcal/mol): |
|---|---|
| TIM009: | -6.8 |
| TIM050: | -8.3 |
| TIM183: | -8.7 |
| TIM207: | -9.6 |
| TIM218: | -8.4 |

We report here the most potent TREX1 inhibitors to date, along with information about their specificity, drug-likeness, amenability to high-throughput chemistry, and mechanism(s) of inhibition.

H-NMR Validation of Leads: All NMR spectra were obtained using a Bruker Ascend 400 MHz NMR equipped with a 60 place autosampler and a 5 mm Prodigy BBO probe cooled with liquid nitrogen. NMR spectra were analyzed using MNova v.14 from MestreLabs. All NMR spectra were obtained using deuterated solvents, and referenced to tetramethylsilane (TMS) as an internal standard.

### REFERENCES

1. Desmet, C. J. & Ishii, K. J. Nucleic acid sensing at the interface between innate and adaptive immunity in vaccination. Nat. Rev. Immunol. 12, 479-491 (2012).

2. Wu, J. et al. Cyclic-GMP-AMP Is An Endogenous Second Messenger in Innate Immune Signaling by Cytosolic DNA. Science 339, (2013).

3. Diner, E. J. et al. The innate immune DNA sensor cGAS produces a non-canonical cyclic-di-nucleotide that activates human STING. Cell Rep. 3, 1355-1361 (2013).

4. Ablasser, A. et al. cGAS produces a 2′-5′-linked cyclic dinucleotide second messenger that activates STING. Nature 498, 380-384 (2013).

5. Tsuchiya, Y., Jounai, N., Takeshita, F., Ishii, K. J. & Mizuguchi, K. Ligand-induced Ordering of the C-terminal Tail Primes STING for Phosphorylation by TBK1. EBioMedicine 9, 87-96 (2016).

6. Chen, Q., Sun, L. & Chen, Z. J. Regulation and function of the cGAS-STING pathway of cytosolic DNA sensing. Nat. Immunol. 17, 1142-1149 (2016).

7. Hervas-Stubbs, S. et al. Direct Effects of Type I Interferons on Cells of the Immune System. Clin. Cancer Res. 17, 2619-2627 (2011).

8. Deng, L. et al. STING-dependent Cytosolic DNA Sensing Promotes Radiation-induced Type I interferon-dependent Antitumor Immunity in Immunogenic Tumors. Immunity 41, 843-852 (2014).

9. Woo, S.-R. et al. STING-Dependent Cytosolic DNA Sensing Mediates Innate Immune Recognition of Immunogenic Tumors. Immunity 41, 830-842 (2014).

10. Mazur, D. J. & Perrino, F. W. Excision of 3′ Termini by the Trex1 and TREX2 3′→5′ Exonucleases CHARACTERIZATION OF THE RECOMBINANT PROTEINS. J. Biol. Chem. 276, 17022-17029 (2001).

11. Orebaugh, C. D. et al. The TREX1 C-terminal Region Controls Cellular Localization through Ubiquitination. J. Biol. Chem. 288, 28881-28892 (2013).

12. Silva, U. de et al. The Crystal Structure of TREX1 Explains the 3′ Nucleotide Specificity and Reveals a Polyproline II Helix for Protein Partnering. J. Biol. Chem. 282, 10537-10543 (2007).

13. Mazur, D. J. & Perrino, F. W. Structure and Expression of the TREX1 and TREX2 3′→5′ Exonuclease Genes. J. Biol. Chem. 276, 14718-14727 (2001).

14. Hasan, M. et al. Cytosolic nuclease TREX1 regulates oligosaccharyltransferase activity independent of nuclease activity to suppress immune activation. Immunity 43, 463-474 (2015).

15. Richards, A. et al. C-terminal truncations in human 3′-5′ DNA exonuclease TREX1 cause autosomal dominant retinal vasculopathy with cerebral leukodystrophy. Nat. Genet. 39, 1068-1070 (2007).

16. Wolf, C. et al. RPA and Rad51 constitute a cell intrinsic mechanism to protect the cytosol from self DNA. Nat. Commun. 7, (2016).

17. Orebaugh, C. D., Fye, J. M., Harvey, S., Hollis, T. & Perrino, F. W. The TREX1 Exonuclease R114H Mutation in Aicardi-Goutières Syndrome and Lupus Reveals Dimeric Structure Requirements for DNA Degradation Activity◆. J. Biol. Chem. 286, 40246-40254 (2011).

18. Fye, J. M., Coffin, S. R., Orebaugh, C. D., Hollis, T. & Perrino, F. W. The Arg-62 residues of the TREX1 exonuclease act across the dimer interface contributing to catalysis in the opposing protomers. J. Biol. Chem. 289, 11556-11565 (2014).

19. Bailey, S. L., Harvey, S., Perrino, F. W. & Hollis, T. Defects in DNA degradation revealed in crystal structures of TREX1 exonuclease mutations linked to autoimmune disease. DNA Repair 11, 65-73 (2012).

20. Yang, Y.-G., Lindahl, T. & Barnes, D. E. Trex1 Exonuclease Degrades ssDNA to Prevent Chronic Checkpoint Activation and Autoimmune Disease. Cell 131, 873-886 (2007).

21. Lindahl, T., Barnes, D. E., Yang, Y.-G. & Robins, P. Biochemical properties of mammalian TREX1 and its association with DNA replication and inherited inflammatory disease. Biochem. Soc. Trans. 37, 535-538 (2009).

22. DiFrancesco, J. C. et al. TREX1 C-terminal frameshift mutations in the systemic variant of retinal vasculopathy with cerebral leukodystrophy. Neurol. Sci. 36, 323-330 (2015).

23. Hemphill, W. O. & Perrino, F. W. Measuring TREX1 and TREX2 exonuclease activities. Methods Enzymol. 625, 109-133 (2019).

24. Belyakova, N. V. et al. Proof-reading 3′-->5′ exonucleases isolated from rat liver nuclei. Eur. J. Biochem. 217, 493-500 (1993).

25. Perrino, F. W., Miller, H. & Ealey, K. A. Identification of a 3′-->5′-exonuclease that removes cytosine arabinoside monophosphate from 3′ termini of DNA. J. Biol. Chem. 269, 16357-16363 (1994).

26. Harrigan, J. A. et al. WRN exonuclease activity is blocked by DNA termini harboring 3′ obstructive groups. Mech. Ageing Dev. 128, 259-266 (2007).

27. Rice, G. I., Rodero, M. P. & Crow, Y. J. Human Disease Phenotypes Associated With Mutations in TREX1. J. Clin. Immunol. 35, 235-243 (2015).

28. Rego, S. L. et al. TREX1 D18N mice fail to process erythroblast DNA resulting in inflammation and dysfunctional erythropoiesis. Autoimmunity 51, 333-344 (2018).

29. Sakai, T. et al. DNase-active TREX1 frame-shift mutants induce serologic autoimmunity in mice. J. Autoimmun. 81, 13-23 (2017).

30. Grieves, J. L. et al. Exonuclease TREX1 degrades double-stranded DNA to prevent spontaneous lupus-like inflammatory disease. Proc. Natl. Acad. Sci. U. S. A. 112, 5117-5122 (2015).

31. Morita, M. et al. Gene-Targeted Mice Lacking the Trex1 (DNase III) 3′→5′ DNA Exonuclease Develop Inflammatory Myocarditis. Mol. Cell. Biol. 24, 6719-6727 (2004).

32. Stetson, D. B., Ko, J. S., Heidmann, T. & Medzhitov, R. Trex1 prevents cell-intrinsic initiation of autoimmunity. Cell 134, 587-598 (2008).

33. Gray, E. E., Treuting, P. M., Woodward, J. J. & Stetson, D. B. cGAS is required for lethal autoimmune disease in the Trex1-deficient mouse model of Aicardi-Goutieres Syndrome. J. Immunol. Baltim. Md 1950 195, 1939-1943 (2015).

34. Gall, A. et al. Autoimmunity initiates in non-hematopoietic cells and progresses via lymphocytes in an interferon-dependent autoimmune disease. Immunity 36, 120-131 (2012).

35. Gao, P. et al. Cyclic [G(2′,5′)pA(3′,5′)p] Is the Metazoan Second Messenger Produced by DNA-Activated Cyclic GMP-AMP Synthase. Cell 153, 1094-1107 (2013).

36. Coscoy, L. & Raulet, D. H. DNA mismanagement leads to immune system oversight. Cell 131, 836-838 (2007).

37. Wang, C.-J., Lam, W., Bussom, S., Chang, H.-M. & Cheng, Y.-C. TREX1 acts in degrading damaged DNA from drug-treated tumor cells. DNA Repair 8, 1179-1189 (2009).

38. Vanpouille-Box, C., Formenti, S. C. & Demaria, S. TREX1 dictates the immune fate of irradiated cancer cells. OncoImmunology 6, e1339857 (2017).

39. Yamazaki, T. & Galluzzi, L. TREX1 Cuts Down on Cancer Immunogenicity. Trends Cell Biol. 27, 543-545 (2017).

40. Alberti, A. S. et al. Engineered trivalent immunogen adjuvanted with a STING agonist confers protection against Trypanosoma cruzi infection. Npj Vaccines 2, 9 (2017).

41. Weiss, J. M. et al. The STING agonist DMXAA triggers a cooperation between T lymphocytes and myeloid cells that leads to tumor regression. Oncoimmunology 6, (2017).

42. Adli, A. D. F., Jahanban-Esfahlan, R., Seidi, K., Samandari-Rad, S. & Zarghami, N. An overview on Vadimezan (DMXAA): The vascular disrupting agent. Chem. Biol. Drug Des. 91, 996-1006 (2018).

43. Gao, P. et al. Structure-Function Analysis of STING Activation by c[G(2′,5′)pA(3′,5′)p] and Targeting by Antiviral DMXAA. Cell 154, 748-762 (2013).

44. Shih, A. Y., Damm-Ganamet, K. L. & Mirzadegan, T. Dynamic Structural Differences between Human and Mouse STING Lead to Differing Sensitivity to DMXAA. Biophys. J. 114, 32-39 (2018).

45. Qian, J. Discovery of small-molecule compounds targeting TREX1 :: University of Southern California Dissertations and Theses. digitallibrary.usc.edu/cdm/ref/collection/p15799coll3/id/74180 (2012).

46. Polishchuk, P. G., Madzhidov, T. I. & Varnek, A. Estimation of the size of drug-like chemical space based on GDB-17 data. J. Comput. Aided Mol. Des. 27, 675-679 (2013).

47. Drew, K. L. M., Baiman, H., Khwaounjoo, P., Yu, B. & Reynisson, J. Size estimation of chemical space: how big is it? J. Pharm. Pharmacol. 64, 490-495 (2012).

48. Reymond, J.-L. The Chemical Space Project. Acc. Chem. Res. 48, 722-730 (2015).

49. McGovern, S. L., Caselli, E., Grigorieff, N. & Shoichet, B. K. A Common Mechanism Underlying Promiscuous Inhibitors from Virtual and High-Throughput Screening. J. Med. Chem. 45, 1712-1722 (2002).

50. Cournia, Z. et al. Discovery of Human Macrophage Migration Inhibitory Factor (MIF)-CD74 Antagonists via Virtual Screening. J. Med. Chem. 52, 416-424 (2009).

51. Tao, H., Yan, X., Zhu, K. & Zhang, H. Discovery of Novel PRMT5 Inhibitors by Virtual Screening and Biological Evaluations. Chem. Pharm. Bull. (Tokyo) 67, 382-388 (2019).

52. Ochoa, R., Rodriguez, C. A. & Zuluaga, A. F. Prediction of Ligands Binding Acetylcholinesterase with Potential Antidotal Activity: A Virtual Screening Approach. Mol. Inform. (2019) doi:10.1002/minf.201800126.

53. Ripphausen, P., Nisius, B. & Bajorath, J. State-of-the-art in ligand-based virtual screening. Drug Discov. Today 16, 372-376 (2011).

54. Lionta, E., Spyrou, G., Vassilatis, D. K. & Cournia, Z. Structure-Based Virtual Screening for Drug Discovery: Principles, Applications and Recent Advances. Curr. Top. Med. Chem. 14, 1923-1938 (2014).

55. Mullard, A. Supersized virtual screening offers potent leads. Nat. Rev. Drug Discov. 18, 243 (2019).

56. Stumpfe, D., Ripphausen, P. & Bajorath, J. Virtual compound screening in drug discovery. Future Med. Chem. 4, 593-602 (2012).

57. Guido, R. V. C., Oliva, G. & Andricopulo, A. D. Virtual screening and its integration with modern drug design technologies. Curr. Med. Chem. 15, 37-46 (2008).

58. Chang, M. W., Ayeni, C., Breuer, S. & Torbett, B. E. Virtual Screening for HIV Protease Inhibitors: A Comparison of AutoDock 4 and Vina. PLOS ONE 5, e11955 (2010).

59. Trott, O. & Olson, A. J. AutoDock Vina: improving the speed and accuracy of docking with a new scoring function, efficient optimization and multithreading. J. Comput. Chem. 31, 455-461 (2010).

60. Morris, G. M. et al. AutoDock4 and AutoDockTools4: Automated Docking with Selective Receptor Flexibility. J. Comput. Chem. 30, 2785-2791 (2009).

61. O′Boyle, N. M. et al. Open Babel: An open chemical toolbox. J. Cheminformatics 3, 33 (2011).

62. McGovern, S. L., Helfand, B. T., Feng, B. & Shoichet, B. K. A Specific Mechanism of Nonspecific Inhibition. J. Med. Chem. 46, 4265-4272 (2003).

63. Babaoglu, K. et al. Comprehensive Mechanistic Analysis of Hits from High-Throughput and Docking Screens against β-Lactamase. J. Med. Chem. 51, 2502-2511 (2008).

64. Auld, D. S. Assay Interference by Aggregation. 33.

65. Medina-Franco, J. L., Petit, J. & Maggiora, G. M. Hierarchical Strategy for Identifying Active Chemotype Classes in Compound Databases. Chem. Biol. Drug Des. 67, 395-408 (2006).

66. Pérez-Villanueva, J. et al. CASE Plots for the Chemotype-Based Activity and Selectivity Analysis: A CASE Study of Cyclooxygenase Inhibitors: Selectivity Analysis of Cyclooxygenase Inhibitors. Chem. Biol. Drug Des. 80, 752-762 (2012).

67. Lyu, J. et al. Ultra-large library docking for discovering new chemotypes. Nature 566, 224-229 (2019).

68. Silicos-it | Strip-it™. silicos-it.be.s3-website-eu-west-1.amazonaws.com/software/strip-it/1.0.2/strip-it.html.

69. Ganesh, A. N., Donders, E. N., Shoichet, B. K. & Shoichet, M. S. Colloidal aggregation: from screening nuisance to formulation nuance. Nano Today 19, 188-200 (2018).

70. Coan, K. E. D. & Shoichet, B. K. Stability and equilibria of promiscuous aggregates in high protein milieus. Mol. Biosyst. 3, 208-213 (2007).

71. Arias, D. G., Regner, E. L., Iglesias, A. A. & Guerrero, S. A. Entamoeba histolytica thioredoxin reductase: Molecular and functional characterization of its atypical properties. Biochim. Biophys. Acta BBA - Gen. Subj. 1820, 1859-1866 (2012).

72. Mazur, D. J. & Perrino, F. W. Identification and Expression of the TREX1 and TREX2 cDNA Sequences Encoding Mammalian 3′→5′ Exonucleases. J. Biol. Chem. 274, 19655-19660 (1999).

73. Parra, D. et al. Increased Susceptibility to Skin Carcinogenesis in TREX2 Knockout Mice. Cancer Res. 69, 6676-6684 (2009).

74. Manils, J. et al. Multifaceted role of TREX2 in the skin defense against UV-induced skin carcinogenesis. Oncotarget 6, (2015).

75. Lipinski, C. A., Lombardo, F., Dominy, B. W. & Feeney, P. J. Experimental and computational approaches to estimate solubility and permeability in drug discovery and development settings1PII of original article: S0169-409X(96)00423-1. The article was originally published in Advanced Drug Delivery Reviews 23 (1997) 3-25.1. Adv. Drug Deliv. Rev. 46, 3-26 (2001).

76. Strelow, J. et al. Mechanism of Action Assays for Enzymes. in Assay Guidance Manual (eds. Sittampalam, G. S. et al.) (Eli Lilly & Company and the National Center for Advancing Translational Sciences, 2004).

77. Kunze, K. Enzyme Kinetics and Reversible Inhibition. 17 (2013).

78. Williams, J. W. & Morrison, J. F. [17] The kinetics of reversible tight-binding inhibition. in Methods in Enzymology vol. 63 437-467 (Academic Press, 1979).

79. Yung-Chi, C. & Prusoff, W. H. Relationship between the inhibition constant (KI) and the concentration of inhibitor which causes 50 per cent inhibition (I50) of an enzymatic reaction. Biochem. Pharmacol. 22, 3099-3108 (1973).

80. Pettersen, E. F. et al. UCSF Chimera--a visualization system for exploratory research and analysis. J. Comput. Chem. 25, 1605-1612 (2004).

81. Deac Wiki. wiki.deac.wfu.edu/user/Main_Page.

82. Silicos-it | Align-it™. silicos-it.be.s3-website-eu-west-1.amazonaws.com/software/align-it/1.0.4/align-it.html.

The foregoing is illustrative of the present invention, and is not to be construed as limiting thereof. The invention is defined by the following claims, with equivalents of the claims to be included therein.

We claim:

1. A method of treating a cancer, viral infection, or autoimmune disease in a subject in need thereof, comprising: administering to the subject a therapeutically effective amount of a compound of Formula I, a compound of Formula II, a compound of Formula III, a compound of Formula IV, or a compound of Formula V: 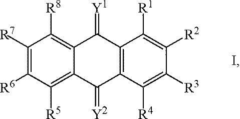 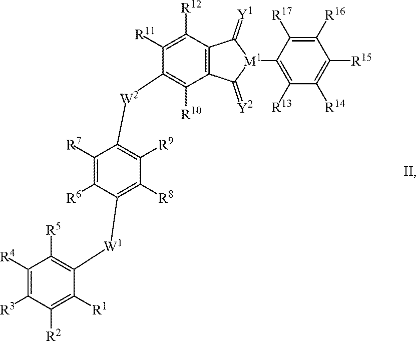 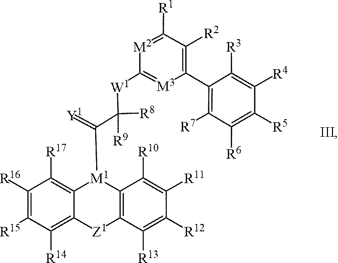 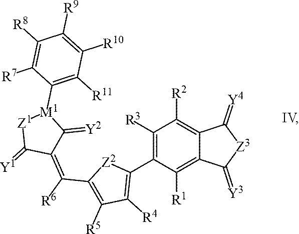 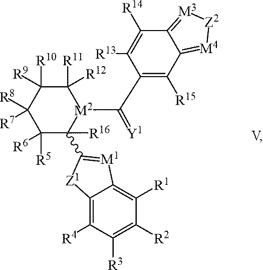
wherein:
M¹, M², M³, and M⁴ are each independently N or C-R′, wherein R′ is H, OH, C₁₋₅ alkyl, halogen, sulfonate, carboxy, C₁₋₅alkoxy, or nitrile;
W¹, W², Y¹, Y², Y³ and Y⁴ are each independently O or S;
Z¹, Z², and Z³ are each independently selected from O, S, N-R″, wherein R″ is H or optionally substituted alkyl, and CR^{a}R^{b}, wherein R^{a} and R^{b} are each independently selected from H, OH, alkoxy, C₁₋₅ alkyl, halogen, sulfonate, carboxy, C₁₋₅alkoxy, and nitrile; and
R¹ to R¹⁷ are each independently selected from H, OH, C₁₋₅ alkyl, halogen, sulfonate, carboxy, C₁₋₅alkoxy, and nitrile,
or a pharmaceutically acceptable salt or prodrug thereof.

2. The method of claim 1, wherein said compound is a compound of Formula I: 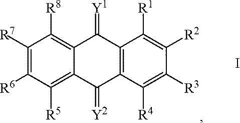
wherein:
Y¹ and Y² are each O;
R¹ and R² are each hydroxy;
R³ is sulfonate
R⁴ is H; and
R⁵, R⁶, R⁷, and R⁸ are each independently H or C₁₋₅alkyl,
or a pharmaceutically acceptable salt or prodrug thereof.

3. The method of claim 1, wherein said compound is a compound of Formula II: 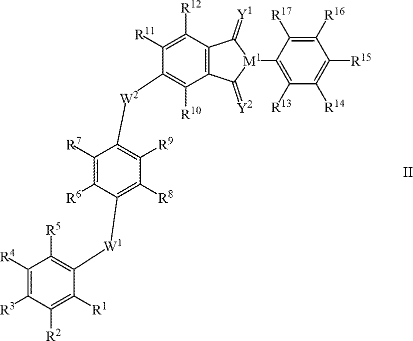
wherein:
Y¹ and Y² are each O;
W¹ and W² are each O;
M¹ is N;
one of R¹ to R⁵ is carboxy and the others are H;
R⁶ to R¹⁴, R¹⁶ and R¹⁷ are each independently H or C₁₋₅alkyl; and
R¹⁵ is alkoxy (e.g. methoxy),
or a pharmaceutically acceptable salt or prodrug thereof.

4. The method of claim 1, wherein said compound is a compound of Formula III: 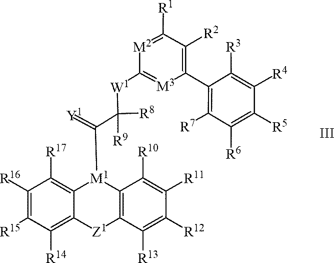
wherein:
Y¹ is O;
W¹ is S;
M¹ is N;
Z¹ is S;
M² and M³ are each N;
R¹ is hydroxyl;
R² is nitrile; and
R³ to R¹⁷ are each independently H or C₁₋₅alkyl,
or a pharmaceutically acceptable salt or prodrug thereof.

5. The method of claim 1, wherein said compound is a compound of Formula IV: 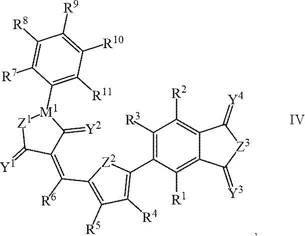
wherein:
Y¹, Y², Y³ and Y⁴ are each O;
Z¹ is NH;
M¹ is N;
Z² is O;
Z³ is NH;
R¹ to R⁸ and R¹¹ are each independently H or C₁₋₅alkyl; and
R⁹ and R¹⁰ are each C₁₋₅alkyl (e.g. methyl),
or a pharmaceutically acceptable salt or prodrug thereof.

6. The method of claim 1, wherein said compound is a compound of Formula V: 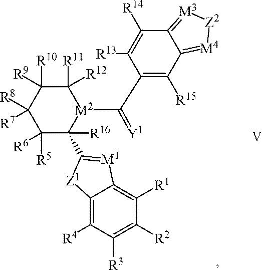
wherein:
Y¹ is O;
Z¹ is NH;
M¹ is N;
M², M³, and M⁴ are each N;
Z² is O;
one of R¹ to R⁴ is halo (e.g. fluoro) and the others are H; and
R⁵ to R¹⁶ are each independently H or C₁₋₅alkyl,
or a pharmaceutically acceptable salt or prodrug thereof.

7. The method of claim 1, wherein said compound is selected from the group consisting of: 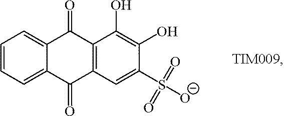 [SMILES: O=C1c2ccccc2C(=O)c2c1cc(S(=O)(=O)[O-])c(O)c2O] 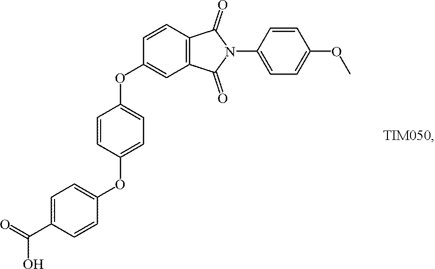 [SMILES: COc1ccc(N2C(=O)c3ccc(Oc4ccc(Oc5ccc(C(=O)O)cc5)cc4)cc3C2=O)cc1] 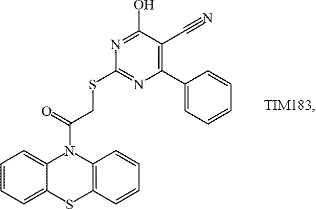 [SMILES: N#Cc1c(O)nc(SCC(=O)N2c3ccccc3Sc3ccccc32)nc1-c1ccccc1] 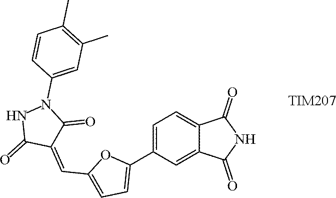 [SMILES: Cc1ccc(N2NC(=O)/C(=C/c3ccc(-c4ccc5c(c4)C(=O)NC5=O)o3)C2=O)cc1C]
, and 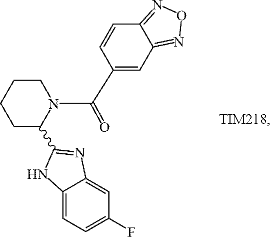 [SMILES: O=C(c1ccc2nonc2c1)N1CCCCC1c1nc2cc(F)ccc2[nH]1]
or a pharmaceutically acceptable salt thereof.

8. A method of treating a cancer, viral infection, or autoimmune disease in a subject in need thereof, comprising: administering to the subject a therapeutically effective amount of a compound of Formula VI, a compound of Formula VII, a compound of Formula VIII, or a compound of Formula IX: 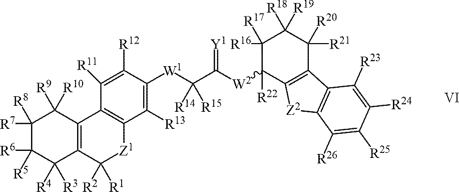 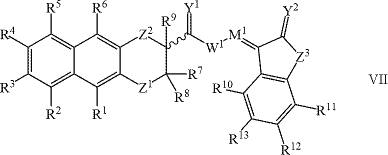 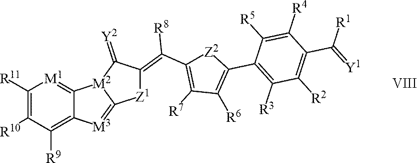 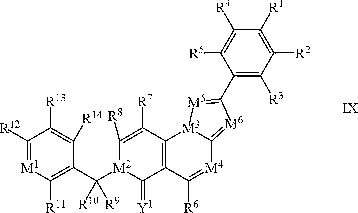
wherein:
M¹ to M⁶ are each independently N or C-R′, wherein R′ is H, OH, C₁₋₅ alkyl, halogen, sulfonate, carboxy, C₁₋₅alkoxy, or nitrile;
W¹, W², Y¹ and Y² are each independently O or S;
Z¹, Z², an Z³ are each independently selected from O, S, N-R″, wherein R″ is H or optionally substituted alkyl, and CR^{a}R^{b}, wherein R^{a} and R^{b} are each independently selected from H, OH, alkoxy, C₁₋₅ alkyl, halogen, sulfonate, carboxy, C₁₋₅alkoxy, and nitrile; and
R¹ to R²⁶ are each independently selected from H, OH, C₁₋₅alkyl, halogen, sulfonate, carboxy, C₁₋₅alkoxy, and nitrile,
or a pharmaceutically acceptable salt or prodrug thereof.

9. The method of claim 8, wherein said compound is a compound of Formula VI: 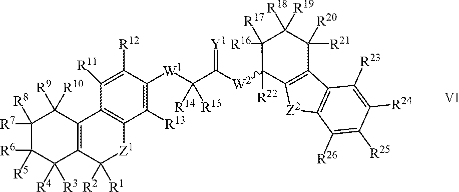
wherein:
Y¹ is O;
Z¹ is O;
Z² is NH;
W¹ is O;
W² is NH;
one of R²³ to R²⁶ is C₁₋₅alkoxy (e.g., methoxy) and the others are each H; and
R¹ to R²² are each H,
or a pharmaceutically acceptable salt or prodrug thereof.

10. The method of claim 8, wherein said compound is a compound of Formula VII: 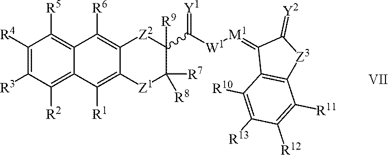
wherein:
W¹ is NH;
Y¹ and Y² are each O;
Z¹ and Z² are each O;
Z³ is NR″, wherein R″ is lower alkyl (e.g. methyl);
M¹ is N;
R¹ to R⁹ are each H; and
one of R¹⁰ to R¹³ is halo (e.g. bromo), and the others are each independently H or lower alkyl,
or a pharmaceutically acceptable salt or prodrug thereof.

11. The method of claim 8, wherein said compound is a compound of Formula VIII: 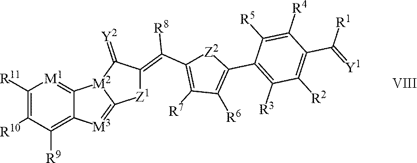
wherein:
Y¹ and Y² are each O;
Z¹ is S;
Z² is O;
M¹, M², and M³ are each N;
R¹ is OH;
one of R⁹ to R¹¹ is halo (e.g. bromo), and the others are each H; and
R² to R⁸ are each H,
or a pharmaceutically acceptable salt or prodrug thereof.

12. The method of claim 8, wherein said compound is a compound of Formula IX: 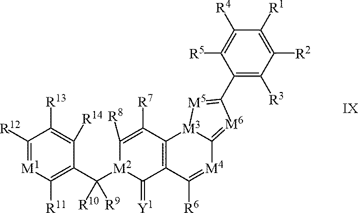
wherein:
Y¹ is O;
M¹ to M⁶ are each N;
one of R¹ to R⁵ is halo (e.g. chloro), and the others are each H; and
R⁶ to R¹⁴ are each H,
or a pharmaceutically acceptable salt or prodrug thereof.

13. The method of claim 8, wherein said compound is selected from the group consisting of: 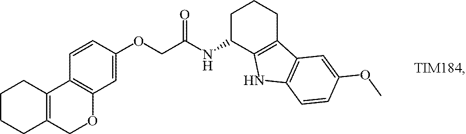 [SMILES: COc1ccc2[nH]c3c(c2c1)CCC[C@H]3NC(=O)COc1ccc2c(c1)OCC1=C2CCCC1] 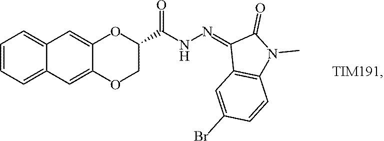 [SMILES: CN1C(=O)/C(=N/NC(=O)[C@@H]2COc3cc4ccccc4cc3O2)c2cc(Br)ccc21] 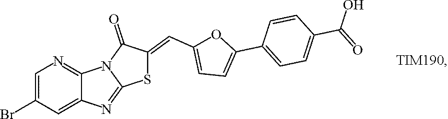 [SMILES: O=C(O)c1ccc(-c2ccc(/C=c3\sc4nc5cc(Br)cnc5n4c3=O)o2)cc1]
and 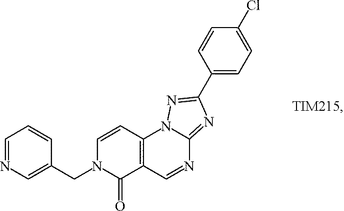 [SMILES: O=c1c2cnc3nc(-c4ccc(Cl)cc4)nn3c2ccn1Cc1cccnc1]
or a pharmaceutically acceptable salt thereof.

14. The method of claim 1, wherein said subject is a human subject.

15. The method of claim 1, wherein said subject is a non-human animal subject (e.g. non-human mammalian subject).

16. The method of claim 1, wherein said administering is carried out by administering a pharmaceutical composition comprising said compound, pharmaceutically acceptable salt or prodrug thereof.

17. A method of inhibiting TREX1 in a subject in need thereof, comprising: administering to said subject a therapeutically effective amount of a compound of Formula I, a compound of Formula II, a compound of Formula III, a compound of Formula IV, or a compound of Formula V, as defined herein, or a pharmaceutically acceptable salt or prodrug thereof, or comprising: administering to said subject a therapeutically effective amount of a compound of Formula VI, a compound of Formula VII, a compound of Formula VIII, or a compound of Formula IX, as defined herein, or a pharmaceutically acceptable salt or prodrug thereof.

18. (canceled)

19. A compound of Formula I, a compound of Formula II, a compound of Formula III, a compound of Formula IV, a compound of Formula V, a compound of Formula VI, a compound of Formula VII, a compound of Formula VIII, or a compound of Formula IX, as defined herein, or a pharmaceutically acceptable salt or prodrug thereof.

20. A pharmaceutical composition comprising a compound, pharmaceutically acceptable salt or prodrug of claim 19.

21. The composition of claim 20, wherein said composition is formulated for oral or parenteral (e.g. intravenous) administration.

22. The composition of claim 21, wherein said composition is formulated for oral administration and is in the form of a capsule, cachet, lozenge, or tablet.

23. The composition of claim 19, wherein said formulation is provided in unit dosage form of from 1 mg to 10 grams of the compound, pharmaceutically acceptable salt or prodrug.

24-29. (canceled)
